SEQUENCE LISTING (65 sequences)

SEQ ID NO: 1 - DNA, 783 bp, Yersinia pestis
  CDS (1)..(783)
atgggtaccaaaaaacgtctgtcggtggtgatgataaccaagaatgaagcctctctgctgacggactgtttggcatccgtggcctgggctgatgaaataatcgtattagactcaggcagtgaagatgaaacacgggcattagctcagcaattcggggcaaaagtttacagcaacatcaattggcccggttacggtaaacagcggcaactcgctcaacaatatgccagcggcgactatattttgatgttagatgctgatgagcgggtgaccccagagcttaaaatcgcgattgaatctgttctgctagcaccagaagagggtgctgtttatagctgttcacgccggaacctctttttagggcgttttatgcgccacagcggctggtatccagatagggtgactcggttatatcctcatcaccaatatcgctacaacgataatttagtccatgaatcactcgatagcggctcagcaaaagttatcccactggctggtgatttattgcatctgacttgccgcgacttttttgcctttcaacgtaaacagcttagttatgccgaagcatgggccatccaacgtcatcagcaaggcaaaagttgcagttattttgccattctcagccatactcttggggcattcagtaaaacgtggttattacgtgctggcttccttgatggtaagcaagggctattactggcggtagtcaacgcgcaatatacttttaataaatatgctgctctgtgggcattgagccaccagtatcagaaaagcgagaaatcatga

SEQ ID NO: 2 - protein, 260 aa, Yersinia pestis
MGTKKRLSVVMITKNEASLLTDCLASVAWADEIIVLDSGSEDETRALAQQFGAKVYSNINWPGYGKQRQLAQQYASGDYILMLDADERVTPELKIAIESVLLAPEEGAVYSCSRRNLFLGRFMRHSGWYPDRVTRLYPHHQYRYNDNLVHESLDSGSAKVIPLAGDLLHLTCRDFFAFQRKQLSYAEAWAIQRHQQGKSCSYFAILSHTLGAFSKTWLLRAGFLDGKQGLLLAVVNAQYTFNKYAALWALSHQYQKSEKS

SEQ ID NO: 3 - DNA, 426 bp, Yersinia pestis
  CDS (1)..(426)
atgcgttatttattatctctgtctgtttttcttatcgtctctcttaatcctgcatttgctgaatggactggtgataacgtagaaggtatgcattcaggcatgattataaataaatttcattcaggccaggttgatgggaaaccttatttctgcattgaggcattcaagccgtcaacaacgataacagcctgttcagtaaaggacacctccatatggggagcatcatataacacgttatatgatcaggctatgtattattatacaaccggtaagcgtatcagagtttattacgctccggatgtctggactaataatagttttgtgagggcacttacagcaaatgctctggttggattctcaacgtgtatctcggaaagttcgtgctttggcccggatagaaaaaaacataaatttaccgtgcattga

SEQ ID NO: 4 - protein, 141 aa, Yersinia pestis
MRYLLSLSVFLIVSLNPAFAEWTGDNVEGMHSGMIINKFHSGQVDGKPYFCIEAFKPSTTITACSVKDTSIWGASYNTLYDQAMYYYTTGKRIRVYYAPDVWTNNSFVRALTANALVGFSTCISESSCFGPDRKKHKFTVH

SEQ ID NO: 5 - DNA, 996 bp, Yersinia pestis
  CDS (1)..(996)
atgaatattctttccctacgcggtggtttttttctgtggatgatgcttatctcaacatcttctttggctattaattgccaaagagcctcaacccccgtcgagtatacagtctgcagcaatgaggatttacactggctggatcaaacatttaacagcatttatcaagtcttattggtgaaatacaatactgaaatggtttatcaacagcgtcaaacatgggaaaaatcgctgaacagttgtaccagtgatagttgtatccagcgggcatatctgcaaggtattgccttaatgtctgatgttgataagaactttaactgggatgggcaatggtggaatatcactaaaggtaatgaccgtggtggggttatcgaaattcatcgagtgaccgagtggggatttagtctggatagccgtgcatggtctggtgagaatatgggtaattttcatgcagaagcccgtaaaacagaaggagtagccgttgttgatgtcattgagaacacggctaattgtcggttattactgatccctggaaaagatggctcgctacaggtgcacagtaatggcagttgggggtgtcgtatctccatgccgaaagatgttttcatcgacgggcaatatgtgcgtgctgaaaaagatccccgtaaggccccaacgttattatctattggtattttttctgaggcttcggtcgatgacgctttccgtaatttagtcggtgagcattatgctcagtttgttcaatctgccaatgtgtatatttacactaatgatttggataaccagggagccaaagtgttatctacctgggtgcggggggcggcgaataagcgtgcttctatcattatgtatacccatactgggcagtattgggcggcttacgttgcaccggataaaaagggtcagttacaaattcactactttaccaatgttgctgcagataaaaataaaaggccaaaaactattgttgaatggcaacaaagttttatagataactga

SEQ ID NO: 6 - protein, 331 aa, Yersinia pestis
MNILSLRGGFFLWMMLISTSSLAINCQRASTPVEYTVCSNEDLHWLDQTFNSIYQVLLVKYNTEMVYQQRQTWEKSLNSCTSDSCIQRAYLQGIALMSDVDKNFNWDGQWWNITKGNDRGGVIEIHRVTEWGFSLDSRAWSGENMGNFHAEARKTEGVAVVDVIENTANCRLLLIPGKDGSLQVHSNGSWGCRISMPKDVFIDGQYVRAEKDPRKAPTLLSIGIFSEASVDDAFRNLVGEHYAQFVQSANVYIYTNDLDNQGAKVLSTWVRGAANKRASIIMYTHTGQYWAAYVAPDKKGQLQIHYFTNVAADKNKRPKTIVEWQQSFIDN

SEQ ID NO: 7 - DNA, 1281 bp, Yersinia pestis
  CDS (1)..(1281)
atggctgacaaaaaagcgacgctgaatttaggcgaagctgcgatcgaactgggtgtcctatccccaaccctcggcactgacgtgatcgacgtccgcaccttaggttctaaaggttattttacctttgaccccggttttacctctaccgcatcctgcgaatcaaaaatcacctatattgatggtgatcagggtattttgctgcaccgtggcttcccgattgcccagttggcaaaagattctacttatctggaagtttgttacattctgctgtacggcgaaacacctacgccagaagagtacaaaacgttcaaaaccacagtgacccgacataccatgatccacgagcagatcacccgtctgttccatggtttccgccgcgattcacatccgatggccgtgctatgtggggttactggtgcgctagccgccttctaccatgatgctctggatgttaataacgagcgccatcgtgaaattaccgccttccgtctgttgtctaaaatgccgactgtcgcagcgatgtgttataaatattcactgggtcaaccgtttgtttatccacgtaacgatctgtcctacgcgggtaacttcctacacatgatgttctctacgccatgtgagaaatatgaagttaacccagtactggaacgcgctatggatcgtattctcatcctgcacgcagatcacgaacagaacgcgtcaacatcgaccgtccgtactgccggctcttctggcgctaacccgtttgcctgtatcgccgcggggatcgcatccctgtggggccctgcgcacggtggtgccaatgaagcctgcctgaagatgttggaagagattaaaactgttgagcatatccctgaatttatccgccgcgcgaaagataagaatgactcattccgtctgatgggttttggtcaccgtgtctataaaaactatgaccctcgtgccaccgtcatgcgtgaaacctgtcatgaagtgttagaagagctgaagctgaataacagcttgttagaagtggcgatggagttggaaaacatcgcactgaacgatccttacttcattgagaaaaaactgtatccaaacgtggacttctattcaggtattatcctgaaagccatggggattccatcgtcaatgtttacggtgatattcgccattgcccgaaccatcggttggattgctcactggaacgaaatgcataatgacggcatcaagattgcccgtccgcgccagctttatactggctatacagaacgtgatttccagagccaattgaaaaaataa

SEQ ID NO: 8 - protein, 426 aa, Yersinia pestis
MADKKATLNLGEAAIELGVLSPTLGTDVIDVRTLGSKGYFTFDPGFTSTASCESKITYIDGDQGILLHRGFPIAQLAKDSTYLEVCYILLYGETPTPEEYKTFKTTVTRHTMIHEQITRLFHGFRRDSHPMAVLCGVTGALAAFYHDALDVNNERHREITAFRLLSKMPTVAAMCYKYSLGQPFVYPRNDLSYAGNFLHMMFSTPCEKYEVNPVLERAMDRILILHADHEQNASTSTVRTAGSSGANPFACIAAGIASLWGPAHGGANEACLKMLEEIKTVEHIPEFIRRAKDKNDSFRLMGFGHRVYKNYDPRATVMRETCHEVLEELKLNNSLLEVAMELENIALNDPYFIEKKLYPNVDFYSGIILKAMGIPSSMFTVIFAIARTIGWIAHWNEMHNDGIKIARPRQLYTGYTERDFQSQLKK

SEQ ID NO: 9 - DNA, 930 bp, Yersinia pestis
  CDS (1)..(930)
atgccacaagttaataacatatcgacaaacaatatacactcagccggctttaacaactcaaattcaattcaaaaatatactggagcagtctcatctataagtgatgatttacgtattaataatgaaaagtgcaagagtgacataggaactataagtggtgatataaaaattaatcgccattctgcagtctacggtaatgtgaattcagtcagtggcgatattactgtaaaaaattctatagtagataaagacatcaccacagtaagtggtgatgttaacgcggtgaattcgactataggaaaaaatataaaaacagtcagtggttcgattgaggtggaacaatcaactgtaagcggcaatttggaaacaacaagtggtgggatagatatagatacaacaaaaattaacggtaatgtgcatacgacaagtggttcaatttcgatgaacgattcaactattgatggtagcgtaacatgtaaggcggggtctgtaacaattgtcaattcaacaattaaagaaagccttaatgtaaccagtgagaaaataattgttggtacagcatcttgtattggaaaaattaacatttccccccctgaatctgtaaattttaacatcatgaattttgggaacgatagtatagtgatgggaatgaggaatttttgtatttcaggagaggtaaattttaccattactaatggtaaagtatttgttaatgaacaaagggtcggccatacagcttcgcaatcaacttcgaaaaaagtagaagaagttactattaatatcgcgaaaaatgccagtgttaatgatattgttttttatactaaaaagtgccatatcattttagaaggaaatgctaaatataacggagaaaaaaaggatggcatgcaattcacccatgttaacgcaccgaaaagtcatgcttatgcgtaa

SEQ ID NO: 10 - protein, 309 aa, Yersinia pestis
MPQVNNISTNNIHSAGFNNSNSIQKYTGAVSSISDDLRINNEKCKSDIGTISGDIKINRHSAVYGNVNSVSGDITVKNSIVDKDITTVSGDVNAVNSTIGKNIKTVSGSIEVEQSTVSGNLETTSGGIDIDTTKINGNVHTTSGSISMNDSTIDGSVTCKAGSVTIVNSTIKESLNVTSEKIIVGTASCIGKINISPPESVNFNIMNFGNDSIVMGMRNFCISGEVNFTITNGKVFVNEQRVGHTASQSTSKKVEEVTINIAKNASVNDIVFYTKKCHIILEGNAKYNGEKKDGMQFTHVNAPKSHAYA

SEQ ID NO: 11 - DNA, 474 bp, Yersinia pestis
  CDS (1)..(474)
atgatcaacaacgatgtgctccgcagtgttcgctatatgcttaacgttaacgataccaaaatagcagaaatcattaagctggctgattttgaagtgaacaacgcagatgtcgttaattttcttaaaaaagaagacgaagccggttatcaggattgccctgatctggtgatggcgcattttctgaatggtttgattttctttagacgtggtaaagatgataagttccctgcacctgcggtagaggcggttatcaccaacaatattgtgttgaaaaaactgcgtgtcgcattcgaattgaaagacaccgatatgcatgatgtttttaatgccgttgaattcccagtctctaagcctgagttaaatgctctgttccgtaaagagggcagcaaaaacttccgcccttgtggtgatcaggttttacgttattttctgaagggcctgacactgcgtattcgtggcgctaaaaaagcctag

SEQ ID NO: 12 - protein, 157 aa, Yersinia pestis
MINNDVLRSVRYMLNVNDTKIAEIIKLADFEVNNADVVNFLKKEDEAGYQDCPDLVMAHFLNGLIFFRRGKDDKFPAPAVEAVITNNIVLKKLRVAFELKDTDMHDVFNAVEFPVSKPELNALFRKEGSKNFRPCGDQVLRYFLKGLTLRIRGAKKA

SEQ ID NO: 13 - DNA, 675 bp, Yersinia pestis
  CDS (1)..(675)
atgaatgaaaattggaattggtctttagtagacaaagttgtttacattaatttgaaggaacgtacggatagaaatgagcacatcaaaaaagaactggaaaaagtgtgctttccacctgaaaaaataatccgctttgaggcaataagagcagggtcaggttttatcggttgcgcaaaatcacatcttgcagtattaaaaatggcgcaagagaataattggagaaatattttgatacttgaagacgatatggtcttcgaagatgatgatgaaacaattatccggacaaacaattttctctctaaactgaataatattcattgggatgctgcatttttatccgccagctattatatcgttaacgctattgatgataatttttttaaagtcaatttcgcctatctggctaacagctatctggtcaataatcattattatgaaaaattaatcaataactacacggagtctgttcagcgattaacaaatggcgagtcatcgtcagagtacgggttggattctaactggttgaaaataatgaaaatagacaactggtacggaatttacccggttattggctatcagcgtaccgatatcagtgatattgaatataaagagatcgatcgcacacaccagttcacccgaacattcgataagatgaaagcatacggttcaaaataa

SEQ ID NO: 14 - protein, 224 aa, Yersinia pestis
MNENWNWSLVDKVVYINLKERTDRNEHIKKELEKVCFPPEKIIRFEAIRAGSGFIGCAKSHLAVLKMAQENNWRNILILEDDMVFEDDDETIIRTNNFLSKLNNIHWDAAFLSASYYIVNAIDDNFFKVNFAYLANSYLVNNHYYEKLINNYTESVQRLTNGESSSEYGLDSNWLKIMKIDNWYGIYPVIGYQRTDISDIEYKEIDRTHQFTRTFDKMKAYGSK

SEQ ID NO: 15 - DNA, 1659 bp, Yersinia pestis
  CDS (1)..(1659)
ttgagcttcgctcatgcacagacattgcccgatgtctattcagtggttgaacgaaaactggaaaatgccctaccgctggcggaacacccccattatgatgctcaggccccttatttcgaactgcaccgagaaatcctgatgtttagctcaccagaacgagccaatacgctattaaagaaacttgatttcagttcgaaagaggcaatgttatcactgaatatttctgctgactggattgcgggaacaggtaatccagacaaagcaatggtatttttatcacagattggactcgagaaaccgagctcattctcagcatacaagaattatgttgatgcctggattgaaaagaaacagcctgaatctgcgttaaagttacttatgctggataacagtgcaaggaactattatcttcccgctgttttagacgcttatcgagatacgccagatcaagcggtgactatttataaagatatttatggcgacaacataatagaaccgaccaatcaattaaggatgctgcttgctattgcagaaaattatcgtgtcaacggtgcccccaaaaacacccttatttataccgataaagcacaggtaatgtttatcgacgtgctcaaaaagaagaagaacaatgaaattcatttttataaagattacttgaatttgatcaatctatattcatttgcaggtaataagcaacaagcattaatattatcagaacagctactcagggcggccggagataaaggtacctattatgatttggcattatccgggatcatggcattttaccatgctaatggtttcactgaagaatacaatgcattaattgcaaccagcattgccactacagataagtcctttagctttgctcccaagcctatagaagaaataaagcttatccggttgttgaatgccactaacgaaaccggcctaattaaagagcgtattgataagctgatgatctcaccagaatatgcctgttatgacgatagatattgctatgaatataaaatagattcgcttaattttctatatttaagcaaatctgatgcattagctgataaatatttaaacattattattgaagaatcacaaaatcaaaaattcaatccatgggaaacggtaactaaatccatcgttaaaaaattagttgatattggacgtatcgccgaagctaagaagttggctgcagatgcagaggttatttatctctcacagctaaaagactctccgcccaaagaagtcgaaagaaattatagggatcttgctgaaatgtatggttttgcaggcgatgtcgtcagcgctgaacgaatacttaataagcatgtcaccaccgcccaaaattactttattaccgatctgtttataaaaaataaacagtgggatgaagccagagcgcgtgttgtgaaagataccggtctttctggacaaaacctgacgttattgcaaaatatttgtgctaccaatacacctgaatgcatgcaacatatcacgttcacacttaaatcaatgttaacacgtgaatccattaccgcagaagatgcatcagggaatcaacaactctaccagttggggatcatctaccactctcttgggataaagccgaccgaggaacaacagcttctcattcaaaaattatatgataatgcggcggcctaa

SEQ ID NO: 16 - protein, 552 aa, Yersinia pestis
LSFAHAQTLPDVYSVVERKLENALPLAEHPHYDAQAPYFELHREILMFSSPERANTLLKKLDFSSKEAMLSLNISADWIAGTGNPDKAMVFLSQIGLEKPSSFSAYKNYVDAWIEKKQPESALKLLMLDNSARNYYLPAVLDAYRDTPDQAVTIYKDIYGDNIIEPTNQLRMLLAIAENYRVNGAPKNTLIYTDKAQVMFIDVLKKKKNNEIHFYKDYLNLINLYSFAGNKQQALILSEQLLRAAGDKGTYYDLALSGIMAFYHANGFTEEYNALIATSIATTDKSFSFAPKPIEEIKLIRLLNATNETGLIKERIDKLMISPEYACYDDRYCYEYKIDSLNFLYLSKSDALADKYLNIIIEESQNQKFNPWETVTKSIVKKLVDIGRIAEAKKLAADAEVIYLSQLKDSPPKEVERNYRDLAEMYGFAGDVVSAERILNKHVTTAQNYFITDLFIKNKQWDEARARVVKDTGLSGQNLTLLQNICATNTPECMQHITFTLKSMLTRESITAEDASGNQQLYQLGIIYHSLGIKPTEEQQLLIQKLYDNAAA

SEQ ID NO: 17 - DNA, 951 bp, Yersinia pestis
  CDS (1)..(951)
atgctcctaaataatattacgccagtgaataaatcactgacactacaagatttattaggaattttgagtcactcatcggcaatttccaatgtggcaaatgggatttatgtggaaagcgaaatccttgaagtaggttcatggctttcagcctacgcggctaataaagatgaaattttttcgcagatcattaccgagttggagaacccttatcaattccagctggagaatgacatacaggcaccgagttttattctttacagtaatgaacgcataactattcgtcttgttatgtggctcccattgcagggaaaattagatcggacaccttattcctacgaagaagcacatgatcataattttgacttttggacagtgaatttttttggaggtggctatcgaactcggctctatgactatgattacgataaagttagtggcgtaaataacgaagttgtagaacttaattgttatggagataagattctttccccgaatacagttatgttttattttcgtagtaaggatgttcatactcaatatccgcctgatgaattatcagtatcgctcaatttaatagtgcgaccaataaaatcgaagcatcaatacgaatttcagatagattcagatgcattggaaggaaaaatagaggcaagaattaaaaagggaagatatgagcgctacgcttttcaaaatgtgttatataacggcctgctgagtcttgaaaatgaaaaaagtcgtcaactggttcacaaagtgtctctttgtaaccatcgagaagagatacgattaatcgcttatgaagctttacttaagcacgcccaaaaaaaaggtaatgtgagtgatataaagagcattagtgaacaagcatttaaagaccaaagcctctatatcaaaaataagatttctcacagtattggaagcatgccatgcatgagtccaaaaccccgctaa

SEQ ID NO: 18 - protein, 316 aa, Yersinia pestis
MLLNNITPVNKSLTLQDLLGILSHSSAISNVANGIYVESEILEVGSWLSAYAANKDEIFSQIITELENPYQFQLENDIQAPSFILYSNERITIRLVMWLPLQGKLDRTPYSYEEAHDHNFDFWTVNFFGGGYRTRLYDYDYDKVSGVNNEVVELNCYGDKILSPNTVMFYFRSKDVHTQYPPDELSVSLNLIVRPIKSKHQYEFQIDSDALEGKIEARIKKGRYERYAFQNVLYNGLLSLENEKSRQLVHKVSLCNHREEIRLIAYEALLKHAQKKGNVSDIKSISEQAFKDQSLYIKNKISHSIGSMPCMSPKPR

SEQ ID NO: 19 - DNA, 1347 bp, Yersinia pestis
  CDS (1)..(1347)
atgaaggttacggtttttggtattggttatgtgggacttgttcaagctacagtgcttgccgaggtggggcatgatgtcctttgtatcgatattgatgctaataaagttgccgatttgaaaaaaggccgtattgctatttttgagccgggcttggctccgttggtcaaagaaaactatgaggcaggcagattacagttttcaacagatgcgcaagctggtgtggcacatggcgtcattcaatttattgctgtgggtacacctcctgatgaagacggtgctgccgatcttaaatatgtccttgatgtggccagtaccattggcacgtatatggactcacctaaggtcattatcgataaatctacagtgcccgtggggacagcggataaagtacatcaagttatacaagctattttggagcagcgcggtgagaagatagattttagtgtggtatcaaatcctgagtttttaaaagagggggccgcagttgctgattgtaaacggcctgaacgtattgttattggtattgatacggatgacagtggtgctctagagttaatcagtgaattatacgaaccttttaatcgaaatcatgatcgtatgctagtaatggatatccgtagtgcagaactgacaaagtatgctgcaaacggcatgctggcaacgaaaataagtttcatgaatgagattgctaatattgctgagcgattaggggctgatatcgaaaaagttcgacagggcatcggttccgattcacgtattggataccatttcatttattctggttgtggttatggggggtcttgtttcccaaaagacatccaagcattaattcgtactgcagaagataagggttataagccccagctattgcaggctgtcgagcaaatcaataatcagcagaagtataagttaactgaattcattaaaagtcattttaattcgcagttagctggaaaaacatttgctgtctggggattggcctttaagcccaacaccgatgatatgcgtgaggcaccgagccgtgtggtaatggaatcattgtgggaagccggggctacaattcaagcatacgatcccgaagcaatggatgagacgcaacgcatttatggtgcaagagatgatttgagattaatggggactaaagagtctgcattacagggagctgatgccttgattatttgtactgaatggcaaagttttcgtgctccagattttgatatgatcaagtcaaggttaaagcaccctgttatttttgatgggcgtaatttgtacgatccagaacgcctaaatagtcgtgggttcacttattatggaattgggcgtggtgtttcgattaatctatcatag

SEQ ID NO: 20 - protein, 448 aa, Yersinia pestis
MKVTVFGIGYVGLVQATVLAEVGHDVLCIDIDANKVADLKKGRIAIFEPGLAPLVKENYEAGRLQFSTDAQAGVAHGVIQFIAVGTPPDEDGAADLKYVLDVASTIGTYMDSPKVIIDKSTVPVGTADKVHQVIQAILEQRGEKIDFSVVSNPEFLKEGAAVADCKRPERIVIGIDTDDSGALELISELYEPFNRNHDRMLVMDIRSAELTKYAANGMLATKISFMNEIANIAERLGADIEKVRQGIGSDSRIGYHFIYSGCGYGGSCFPKDIQALIRTAEDKGYKPQLLQAVEQINNQQKYKLTEFIKSHFNSQLAGKTFAVWGLAFKPNTDDMREAPSRVVMESLWEAGATIQAYDPEAMDETQRIYGARDDLRLMGTKESALQGADALIICTEWQSFRAPDFDMIKSRLKHPVIFDGRNLYDPERLNSRGFTYYGIGRGVSINLS

SEQ ID NO: 21 - DNA, 822 bp, Yersinia pestis
  CDS (1)..(822)
atgaagaatattaaggtttatcaatatatcaagtgggtcattatgccaactattgtgctgttaatgtggcaaatgctcgccatatcgggctggatacaacccacaatacttccatcaccagtagccgttgtggtgcgctggttcgattatttcttaccctatgaagcctataacccagaaacatcaaactggctgatgtggagtctttcaggggagttactgcatgatgcctgggcgagtatctaccgcgtaattattggttttctaatcggtgctcttcttgctctaccattaggattatgggtgggttccagtaaaattggcaatgacctaatgaaccctttaattcagtttcttcgccccattccgccaattgcatatattccattagcgatgctgtggttcggcctggggaatccgccggcattttttttgataagtataggggcttttttcccaatactgattaataccatcgcgggggtccgtaatgtggatggcatatatatcctggctgcacgcaatctaggcgcaggtcaagccacgatgtttcgccgaattattcttccggcggccactccttatattcttacagggatgcgagttggtatagggaccgcgtttatttgcgtgattgtggcagaaatgatcgccgttaatagcggtgttggctaccgaattctggaagcgcgcgaatatatgtggtccgataaagtaatagcaggcatgttcacaattggttttcttggcctggtcattgatttatgcatggatcgcctgaatcggcgtcttttacgctggcacagaggaataggcaactga

SEQ ID NO: 22 - protein, 273 aa, Yersinia pestis
MKNIKVYQYIKWVIMPTIVLLMWQMLAISGWIQPTILPSPVAVVVRWFDYFLPYEAYNPETSNWLMWSLSGELLHDAWASIYRVIIGFLIGALLALPLGLWVGSSKIGNDLMNPLIQFLRPIPPIAYIPLAMLWFGLGNPPAFFLISIGAFFPILINTIAGVRNVDGIYILAARNLGAGQATMFRRIILPAATPYILTGMRVGIGTAFICVIVAEMIAVNSGVGYRILEAREYMWSDKVIAGMFTIGFLGLVIDLCMDRLNRRLLRWHRGIGN

SEQ ID NO: 23 - DNA, 891 bp, Yersinia pestis
  CDS (1)..(891)
atgagccaccctgtttttgtccgccctgagctggtagtggataacgtcactgtcacttataacaatggtcatactgccatttatgacgccagcttttcactaacgggtgggacgatctgtgcactggtaggtgttaatggcagcggtaaatcgaccttatttaaaagcattatggggttagtcaagcccagtgtgggcaaagtggaactgagtcacaaacctatcagtcatgcactaaaacaaaatacgattgcttatgtaccacagacagaagatgtcgattggaacttccctgtgcttgtggaagatgtcgtgatgatgggccgttacggtaaaatgaattttctacgaattcccagtcgtgaagacaaagccattgtgaataaatccattgaacgagttgggttaacggcattgcgttcacggcaaattggtgaactgtctggtggccagaaaaaacgtgttttcttggcccgagcccttgcccaacaaggcacactcttactactggatgagccatttaccggcgttgacgtcaaaactgaaaatgccattattgagttactgcaatctctacgtgacgaaggccatctgattctggtttcaactcataacctaggcagcgtgccggaattttgtgatcacgtcatcctaattaatcagacggtgctggccgcgggcccaattgaaacgacatttacccagaaaaacctcgagatgacctttggcggggtattgcgccatatcaatctgtcggggacggcactgcatgatgataatgatccgcgcaccgtcaccgtaataactgatgatgaacgccccgcagtattttatggtcatactaaaaatgatccgcctgcacaaagccagtcaaaggagcaaaattcctga

SEQ ID NO: 24 - protein, 296 aa, Yersinia pestis
MSHPVFVRPELVVDNVTVTYNNGHTAIYDASFSLTGGTICALVGVNGSGKSTLFKSIMGLVKPSVGKVELSHKPISHALKQNTIAYVPQTEDVDWNFPVLVEDVVMMGRYGKMNFLRIPSREDKAIVNKSIERVGLTALRSRQIGELSGGQKKRVFLARALAQQGTLLLLDEPFTGVDVKTENAIIELLQSLRDEGHLILVSTHNLGSVPEFCDHVILINQTVLAAGPIETTFTQKNLEMTFGGVLRHINLSGTALHDDNDPRTVTVITDDERPAVFYGHTKNDPPAQSQSKEQNS

SEQ ID NO: 25 - DNA, 213 bp, Yersinia pestis
  CDS (1)..(213)
atgagtttcaagtcaataaataaaaacgcagatttgtacgtgacttgcagtggcctcgaagaggcggggtccatggacgggccgagtcacgaaggcaaccagcacacaggcagcttgaagcctgatgggtatattatcattcaagaaaatgatttaatttctggatttgataacacagtcggcatagctaataaccgtttcaggagatgttaa

SEQ ID NO: 26 - protein, 70 aa, Yersinia pestis
MSFKSINKNADLYVTCSGLEEAGSMDGPSHEGNQHTGSLKPDGYIIIQENDLISGFDNTVGIANNRFRRC

SEQ ID NO: 27 - DNA, 657 bp, Yersinia pestis
  CDS (1)..(657)
atgcagaaagaaaagctttccgctttgatggatggagaaactctcgatagcgagctgctggggattttatcgaaagataaggcgttacagcaaagctggcagagctaccatttaatccgtgacactctacgcggagacgttggcaatgttttacatctggatattgctagtcgtgtagcagaagcgctcaagaacgagccagcgcgcttgattcctattgctatacctgaatctcagccacagccacatctttggcagaaaatgcctttctggcaaaaagtccgtccatgggccagtcagattactcaggtgggaatggccgcttgtgtttctctcgctgtcattgttggtgttcaacaatataaccaaccggcacaaaattatttacaacctgaatcaccggcgtttaataccttgccaatgatgggtaaagcatcaccagtcagttttggtgtccctgctgatggttcttttggtactaatcaacagaatcaggttcaagagcagcgtcggcgcgtcaacatgatgactcaattgcaagattttgaattgcaacgtcgtttgccacaaactgatgtattgcagcagccttcagattcaacgcaagccgcaattcaagtccctggaactcaatccttaggaatgcagcagcagtaa

SEQ ID NO: 28 - protein, 218 aa, Yersinia pestis
MQKEKLSALMDGETLDSELLGILSKDKALQQSWQSYHLIRDTLRGDVGNVLHLDIASRVAEALKNEPARLIPIAIPESQPQPHLWQKMPFWQKVRPWASQITQVGMAACVSLAVIVGVQQYNQPAQNYLQPESPAFNTLPMMGKASPVSFGVPADGSFGTNQQNQVQEQRRRVNMMTQLQDFELQRRLPQTDVLQQPSDSTQAAIQVPGTQSLGMQQQ

SEQ ID NO: 29 - DNA, 1413 bp, Yersinia pestis
  CDS (1)..(1413)
ttgctcatgctattacctgtaattatggctggaggtgctggtagccgtttgtggccattatcccgagctctttatcctaaacaatttctagcgctaacgtcagatttgacgatgctacaagaaaccctattgcgtctggacggccttccccaccttgcaccattagtgatttgtaacgaagaacatcgctttattatcgcagaacagttacgtcagaaaaatctggtgcatagcggaatagtcttggaacctgttgggcgcaataccgcgccagctatagcattggctgccttacgagcaacaatgagtggggatgatcctctattattggtattagcagccgatcacgtgattcaggataaacttgcatttattcgtgccgtccaacgtgctgaaccgcttgctgaagcgggaaaattggttacttttggaatcgtgccaaagagtccggaaacaggatatggatatattcgccaagggaagcaagtcgtagatggcgcttatcaggttgctgcttttgttgagaagccagatctgattactgcagagcggtatttggcttcgggtgactattattggaatagcggtatgtttgtatttaaagcatctcgctatctacaggaattagatctacatcgtccggatattttggctgcctgcaagcaagccattgctggtcaacatactgatttagattttattcgtctcaatgaagaagctttctctagttgccctgatgaatctatcgactatgctgtgatggaaaaaactagcgatgccgttgtagtgccactggatgcacagtggaatgatgttgggtgctggtcagcgctttgggaaattaatactaaagatgaccatggtaatgttattcgtggtgatgtattaatagaagatactaataatagctacgtttattctcaaaataggctcattgcaactgtaggcattaatgatttggttattgttgaaactaaagatgccattttagttgctcataaagataaagtacaaaatgttaaagggatcgttggacagcttaagcttgaatctcgatgtgaatatctacagcaccgggaagtctatcgcccttggggttcgcatgatgctattgctgaaggtgttcgctaccatgtccaacatgtaacggtgaaaccaggtcaacgtattgctactcaaattcattatcacagagcggaacattggattgtggtttctggcattgccaaagtacattatggaaaagaaacttatttggttaatgagaatgaatccacttatattcccgtcggcattgctcattctattgaaaatccaggtcagatccctctggaaataatagaggttcatactggtaattacatttcagaagatgatgtggaacgaatcgatgatttaggtgtagggtattaa

SEQ ID NO: 30 - protein, 470 aa, Yersinia pestis
LLMLLPVIMAGGAGSRLWPLSRALYPKQFLALTSDLTMLQETLLRLDGLPHLAPLVICNEEHRFIIAEQLRQKNLVHSGIVLEPVGRNTAPAIALAALRATMSGDDPLLLVLAADHVIQDKLAFIRAVQRAEPLAEAGKLVTFGIVPKSPETGYGYIRQGKQVVDGAYQVAAFVEKPDLITAERYLASGDYYWNSGMFVFKASRYLQELDLHRPDILAACKQAIAGQHTDLDFIRLNEEAFSSCPDESIDYAVMEKTSDAVVVPLDAQWNDVGCWSALWEINTKDDHGNVIRGDVLIEDTNNSYVYSQNRLIATVGINDLVIVETKDAILVAHKDKVQNVKGIVGQLKLESRCEYLQHREVYRPWGSHDAIAEGVRYHVQHVTVKPGQRIATQIHYHRAEHWIVVSGIAKVHYGKETYLVNENESTYIPVGIAHSIENPGQIPLEIIEVHTGNYISEDDVERIDDLGVGY

SEQ ID NO: 31 - DNA, 1014 bp, Yersinia pestis
  CDS (1)..(1014)
atgaaaattatttatgatggaataattaattcattgcaaaatatgggaggggtaactgtctatttctcggagttagtgagtcgacttcctgcaacagatttttattggtactcatacgcagatgaaatatctggtttcggtgttggtaatgttaaaataaaatctagacttttagagcgatataggaatttctcattagatactattaattataattctctttctgtatttcattcatcatattaccgattaccagattttgacattccgatagttacaacagttcatgatttcacatatgaaaagtttgttaatggccctgcaaaatgggtgcattcctggcagaaaaaccgtgcggttaacaatagtgatttaattatttgtgtttcggaaaatacagctaaagatttgcaaatgtattgctcagttcctagcaataaaatcagagtgatatataacggtgtgtcagataaatatcattatattaagggtgttaaaataaccacaaataaagttatttttgttggtgcacgtggtggttataagaattttgacattgcagtgaaagcaatatcaaaaacacctcacctcgaattatcagttgtaggtggaggggcattcactagtaaagaactgtcactactgaatcactatttacctggtcgctaccatggattaggtcgccttagtgatgaggctttgaatgaggcatataattcagcttatgcgctgctttacccatctagctatgagggttttggtattccaatattagaagcgatgagtgcaggatgtcccgtaatatctgttaatgtatcttctatacctgaggtcgcaggtgatgccgctatattagtgcaaaaaccgactattgatgaactagttgacggcttgcttgccgtagaaagtgaaaggtctaaacttattggctatggcatgaagcaagcggctaaattctcatgggataagtgttatcaagaaaccttagatgtttataaagaattgaacaaataa

SEQ ID NO: 32 - protein, 337 aa, Yersinia pestis
MKIIYDGIINSLQNMGGVTVYFSELVSRLPATDFYWYSYADEISGFGVGNVKIKSRLLERYRNFSLDTINYNSLSVFHSSYYRLPDFDIPIVTTVHDFTYEKFVNGPAKWVHSWQKNRAVNNSDLIICVSENTAKDLQMYCSVPSNKIRVIYNGVSDKYHYIKGVKITTNKVIFVGARGGYKNFDIAVKAISKTPHLELSVVGGGAFTSKELSLLNHYLPGRYHGLGRLSDEALNEAYNSAYALLYPSSYEGFGIPILEAMSAGCPVISVNVSSIPEVAGDAAILVQKPTIDELVDGLLAVESERSKLIGYGMKQAAKFSWDKCYQETLDVYKELNK

SEQ ID NO: 33 - DNA, 990 bp, Yersinia pestis
  CDS (1)..(990)
atgtcattaaatgttaagctgcatccatcaggtattatttttacttccgatggaacatctacaatattagatgcggctctggatagtaatatacatattgaatacagctgcaaagatggaacctgtggttcttgtaaggcaatattgatttctggtgaagtagacagtgcggaaaatacctttttaactgaggaagatgttgctaaaggtgcaatcctcacttgttgctctaaggctaaatctgatattgagttagatgttaattattatccagagttaagtcatatacaaaaaaaaacttatccatgtaaattagatagcattgaatttattggtgaagatattgccattctctccttacgtttgccaccaacggccaaaatacagtatctggcgggccaatacattgatttaattattaatggacagcgccgtagttactctattgctaatgctccaggtggtaatggcaatatcgaattacacgtacgtaaagttgttaatggtgtattcagcaacatcatttttaatgagttaaaattacagcaacttttacgaattgaaggtcctcaagggacctttttcgttcgtgaagataatctccctattgtttttcttgctggtggaacaggttttgcaccagtgaaatcaatggttgaggcgttgatcaataaaaatgaccaacggcaggttcatatctattggggaatgccagcagggcataatttctattctgacattgccaatgagtgggctataaaacaccctaacattcattatgtgcctgttgtatcaggcgatgatagtacttggaccggagccactggttttgtacatcaagcggtgcttgaagatatacccgatctcagcttatttaatgtttatgcctgtggttcattagcgatgattactgctgctcgtaatgatttcatcaatcatggattagctgaaaataaatttttctctgatgcctttgtgccatcaaaataa

SEQ ID NO: 34 - protein, 329 aa, Yersinia pestis
MSLNVKLHPSGIIFTSDGTSTILDAALDSNIHIEYSCKDGTCGSCKAILISGEVDSAENTFLTEEDVAKGAILTCCSKAKSDIELDVNYYPELSHIQKKTYPCKLDSIEFIGEDIAILSLRLPPTAKIQYLAGQYIDLIINGQRRSYSIANAPGGNGNIELHVRKVVNGVFSNIIFNELKLQQLLRIEGPQGTFFVREDNLPIVFLAGGTGFAPVKSMVEALINKNDQRQVHIYWGMPAGHNFYSDIANEWAIKHPNIHYVPVVSGDDSTWTGATGFVHQAVLEDIPDLSLFNVYACGSLAMITAARNDFINHGLAENKFFSDAFVPSK

SEQ ID NO: 35 - DNA, 3583 bp, Yersinia pestis
gtgagattgtttgcacaattaggttggtattttcgccgtgaatggcaccgttatgtaggggcggtgctactactgattattatcgctattctgcaattaattccgcccaagttggtgggcgttatagtggatgatatcagtacaaaacagatgtccaccaatatgttattggtttggattggcgtgatgctcgcgactgccgtggtggtctatttgttgcgttatgtctggcgagtcttattattcggtgcctcttatcagttggcggtggagttgagatctgatttttatcgtcagctgagtcggcaaactcccggtttttattcacgtcatcgcactggcgatttaatggctcgcgccaccaatgatgttgatcgcgttgtcttcgccgcaggtgaaggggtactaacgctggtcgattcactggtcatggggtgtgcggtattgattgttatgagcacccaaatcagctggcaattaacactgttatcgctattaccgatgcccatcatggcgatagtgattaagtattacggtgaccaacttcatcagcgttttaaatccgctcagggtgccttttctttgcttaataatcaggctcaagagagcctgaccagtattcgcatgattaaggcctttggtctggaagatcgtcagtcacaacagtttgctcaggtagcggcagaaaccggtgcgaagaatatgtatgtcgcccgcattgatgcccgctttgaccctacaatttatattgctattggtatcgctaatttattagctattggtggcggtagctggatggtggtgaataacagcattaccttagggcaattaaccagttttgttatgtatttggggctgatgatttggccaatgctggcattggcatggatgtttaatattgttgagcgcggtagtgccgcttatagccgcatccgcagtttgttggatgaagcacctgtggttaaagatggccacattactttgtctgatgtgcgtgacacattagcggttaatattcgtcatttttgttatccaggcagtgatcaaccggcactacataatgtggtactgacgctggttccaggggccatgctggggttatgtgggccaacaggttcgggtaagagtaccttgctggccttaatccagcggcaatttgatattgatgacggtgttatttgttatcaagggcatccgctgtcggatattcggttgaatgattggcgtggccgtttatcggtcgttagccagacccctttcctgttctccgatacggtggccggtaatattgccttgggtaaacccgatgcgacccccgcacagattgagcaggcggcccgcctggcctgtgtacatgaagatattttgcgtctgccgcagggttatgacactgaagtaggcgagcgtggagtgatgttgtccggtggacagaaacagcgcatatctattgccagagcgcttttgttggatgctgaaatacttattcttgatgatgcactttctgcggttgatggccagacggagcatgaaattttaaaaaacctacgcgagtggggcgaacagcgtaccgtcattatcagcgcgcatcgcctttctgcattgactgaagccagcgaaattctggtgatgcaacatggtggtgtaatgcagcgggggccccacagccttctagtcaatcagacgggttggtatcgggagatgtaccgctatcagcaattagaagccgcattggatgacggtgagcaggaggtcgaagccgatgaataacgttcagcaactttggccgacattgaaacgcctgctttcctacggttcgccttatcgtaaaccgctggggctggcagtcttaatgctgtgggttgcggcggcagcagaggtaagtggtccactactgatcagttattttattgaccatgtagtcgccaaaggcacattacctctggggctagtcagtgggttggcattggcctatctgttgttgcaattattggccgcgacattgcactattttcaggcattgctgtttaaccgtgcggcagtgggggtcgttcagcggctacgcattgatgtgatggacgctgcattacgtcaacctctcagtgcttttgatactcagcctgttgggcagttaatttcccgagtaaccaatgacaccgaagtgatcaaagatttatatgtcatggtggtttctacggttttgaaaagtgcggccttaattagtgcgatgctggtggcaatgtttagtctggattggcggatggcgctgatttcaatttgtatcttccctgcggtgttggtggtgatgacaatctatcagcgctatagcacccctatcgttcgccgggtacgatcctatctggctgatattaacgatggttttaatgaagtcattaatggtatgggcgtcattcagcaattccgtcagcaggcccgtttcggagaacgcatggcgtctgccagccgtgcgcattatgttgcccggatgcaaaccttacggctggagggctttctattacgccctttattgagcctattctctgccttggtgctatgtggtctactgctgcttttcggtttcagccctgaaggttctgtgggggtaggtgtgctgtatgcgttcattaattaccttggccgcctgaatgagccgttgattgaactgacatcgcagcaatcaattatgcaacaggccgtagtggcaggagagcgtatttttgatctgatggatcgtgcgcagcagggctatggtagcgacaatattcccttgagcagtggccgtattcaggtggaaaacgtcagctttgcgtatcgctcggataaaatggtgttacacaatatttctctcaatgtcccctcccgtggattcgtggctttagttgggcacaccggcagtggtaaaagtacgctagctaacctgttgatgggctactatcccgttcagcaaggtgagattttgcttgatggccggccattatcccgcctctcccatcaggttctgcgccagggggtagcgttggtacagcaagatcccgtggtgttggctgactccttctttgcgaatatcacccttgggcgtgaccttagtgaacagcaagtgtgggaggcactcgaaaccgttcaattggcaccgttggttcgtaccttacctgatggcttgtacagtttacttggggaacagggcaataccttgtctgttgggcaaaaacagttgctggcgatggcgcgggtgttggtgcaagcgccccaaatcctgattctggatgaagccactgcgaatattgactctggcactgaacaggctattcagcgggcattacaggtgattcgaaaaaataccacgctggtggttattgctcatcgcctttcgacgattgtggaggcggatagtattttggtactgcaccgtggtgttgcggttgagcagggtaatcatcaagcgctgctagctgcccgtgggcgttattaccagatgtaccagttgcagttagtcagcgaagatttggctgccattgatcaggaagctattgataaaggctctattgatcaaagtactattgatcaagccggaatgagcgtgagttaa

SEQ ID NO: 36 - DNA, 624 bp, Yersinia pestis
  CDS (1)..(624)
atgtttaaacgtctattcatggtcgcattgttggccatagcaccactggttcatgctgtcgatcaaagcaacccttatcgtctgatggatgaggcagcgaagaaaacctttacgcgtctgaaaaatgagcagcctaagattaagcaaaatccagattatctccgcaccattgtacgcgaagagctgttgccgtttgttcagatcaaatacgccggcgcgttagtactgggtagctattataaagatgctacacctgcacagcgtgaagcgtatttcaatgcattcggtaaatatctggagcaggcatacgggcaggcattggcgttgtatcacggccaaacttacgatgtggcaccagaccagcctttaggggatgccaacatcgttgctatccgcgtcaccattctcgatccaagtgggcgtccacctgtccggttagatttccaatggcgtaaaaatagccaaaccggtaactggcaggcttatgacatgatcgccgaaggggtgagcatgattagcaccaaacagaatgaatgggcctctatcctgcgccaaaagggtgtggatggtttgacccaacaattgctgagtgcggctaaacagccaatcaccttagataaatag

SEQ ID NO: 37 - protein, 207 aa, Yersinia pestis
MFKRLFMVALLAIAPLVHAVDQSNPYRLMDEAAKKTFTRLKNEQPKIKQNPDYLRTIVREELLPFVQIKYAGALVLGSYYKDATPAQREAYFNAFGKYLEQAYGQALALYHGQTYDVAPDQPLGDANIVAIRVTILDPSGRPPVRLDFQWRKNSQTGNWQAYDMIAEGVSMISTKQNEWASILRQKGVDGLTQQLLSAAKQPITLDK

SEQ ID NO: 38 - DNA, 1455 bp, Yersinia pestis
  CDS (1)..(1455)
atgcaaactgatgtcgttctgccgttagtcggctatctggtgttggtctttggcttatctatttatgcttatacgcgccgccaaaccgggaattttctcaacgaatattttatcggcaaccgttcgatgggcggttttgtattggcgatgaccctgaccgccacctatatcagcgccagctcgtttattggtggaccaggtgccgcttataaatacggccttggctgggtcttactggcgatgatccaattgcctgcggtctggctttccctcggtgtcttaggcaaaaagtttgccattctggcgcgtcgctataatgccgtgaccctcaacgatatgttgtatgcccgttatcagagccgtttactggtttggctggccagtatcagcctgctggtggcttttgtcggtgccatgaccgtgcaatttattggtggcgcacggttgttagaaacggcggcggggatcccttacgacactggcttattgatttttggtatcagtatcgccctgtacacctcgtttggtggcttccgggccagtgtcttgaatgatgccctgcaagggttagtgatgctgatcggcaccattttactgttagttgcggtgatccacgcggcaggtggcctacacaaagccgtcgaaacgctgcaacatatcgatccggcgctggtttccccccaaggcggcgatcagatcctcgacgtgccatttatggcttcgttctggatcctggtctgttttggggtgattggtctgccacacaccgccgtacgttgtatttcgtatcgtgacagtaaagcggttcatcgcggtattatcctcggcaccatcgtggtggcgatcctaatgttcggtatgcacctggctggcgcactggggcgggcggtgttaccagatctgaaaattccagatcaagtaatcccgacactgatgatcaccgtactgccgccatttgctgcggggatattcctggcagcaccgatggcggcgatcatgtcgaccatcaatgcccagttactgcaatcttctgctaccatcgtgaaggatttgtatctcaatctgtggccagcagagttaaagaatgagcgcaagctggcacgtatctccagcttatccacactcattcttgggctattattgctcttggccgcctggcgaccacctgaaatgattatctggttgaacctactggcctttggcggactggaagcggtattcctctggccattggtattgggattatactgggagcgagccaatgcccacggcgctcttagcgccatgatcgtcggtgcggtatgctatacggtattagccagcttcgacatcaagatagccggcctgcaccccattgtgccgtcacttacacttaatctgttggcgttttatatcggtaatctgtttggcgacagagcgcgggcgcgacaccccgccatcgtcagtgccgattaa

SEQ ID NO: 39 - protein, 484 aa, Yersinia pestis
MQTDVVLPLVGYLVLVFGLSIYAYTRRQTGNFLNEYFIGNRSMGGFVLAMTLTATYISASSFIGGPGAAYKYGLGWVLLAMIQLPAVWLSLGVLGKKFAILARRYNAVTLNDMLYARYQSRLLVWLASISLLVAFVGAMTVQFIGGARLLETAAGIPYDTGLLIFGISIALYTSFGGFRASVLNDALQGLVMLIGTILLLVAVIHAAGGLHKAVETLQHIDPALVSPQGGDQILDVPFMASFWILVCFGVIGLPHTAVRCISYRDSKAVHRGIILGTIVVAILMFGMHLAGALGRAVLPDLKIPDQVIPTLMITVLPPFAAGIFLAAPMAAIMSTINAQLLQSSATIVKDLYLNLWPAELKNERKLARISSLSTLILGLLLLLAAWRPPEMIIWLNLLAFGGLEAVFLWPLVLGLYWERANAHGALSAMIVGAVCYTVLASFDIKIAGLHPIVPSLTLNLLAFYIGNLFGDRARARHPAIVSAD

SEQ ID NO: 40 - DNA, 879 bp, Yersinia pestis
  CDS (1)..(879)
atggcgagattttggcagatactgattgcgttattactggtgccaacattggcacaggctgaagaagcaacgattgaaaagattcatgatgcgcccgcagtgcgtggcagtattatcgcggcaatgttgcaggaccacgataatcctttcctactttatccgtatgaaaccaactatttgctctacacctataccaacgagatcaataaacaagccattagctcgtatgattgggctgaacaggcgaataaagatgaagtaaaattccaactgagtttagctttccctatctggcgtggtattgccggggataactcattattgggggcttcttatacccagcgttcatggtggcaggcatccaacagtgaagaatcttcaccttttcgtgaaaccaactacgagccacagctgtttttggcgtggtcaacagattacgaattggctggctggactttccgcgaagtcgaatttggttttaaccatcaatccaacggtaaagctgacccaacctcacgtagttggaaccgtgtttatacccgcgtgatggcacaacgtggcaatttggaaatcgacctgaagccttggtatcgtattcctgaaagcgacagtaaagatgataaccctgatattacgaaatacatggggtattatcgcctgaaagtcggctatgccctgggtgacagcgtattcagccttgatggtcgctataactggaataccggttacggtggcgcggaaatgggctggagctacccaattaccaaacatgttcgcttctatactcaggtattcagtggctacggtgagtcaatgattgactataactttaggcaaacacgggtgggtgtaggtatcatgttgaacgatgtcctttaa

SEQ ID NO: 41 - protein, 292 aa, Yersinia pestis
MARFWQILIALLLVPTLAQAEEATIEKIHDAPAVRGSIIAAMLQDHDNPFLLYPYETNYLLYTYTNEINKQAISSYDWAEQANKDEVKFQLSLAFPIWRGIAGDNSLLGASYTQRSWWQASNSEESSPFRETNYEPQLFLAWSTDYELAGWTFREVEFGFNHQSNGKADPTSRSWNRVYTRVMAQRGNLEIDLKPWYRIPESDSKDDNPDITKYMGYYRLKVGYALGDSVFSLDGRYNWNTGYGGAEMGWSYPITKHVRFYTQVFSGYGESMIDYNFRQTRVGVGIMLNDVL

SEQ ID NO: 42 - DNA, 2556 bp, Yersinia pestis
  CDS (1)..(2556)
atggagcaaccccgcctctccttctttgccagtgttcgtgggcgattgttgttcttcaacttgctcgtggtggcggtcaccctgatggtgagtggtgtcgcggtgctgggcttcgagcaggcaagccgcttacaaaagcaggtccaggagcggacactgcgcgatatgtcaagcagcatggcgctggcgcgcgataccgcaaatgtggcgacggcggcggtgcggctttcccaagtggttggggcgcttgaattccagagtgaagcggccagtcttcaagagacgcaactggcgttacgaagttcactcgctcatcttgctaatgccccgcttgccagccatgaaccgctattggtgaaacgtatcattgagcgtagcaatgaactggaaaccagcgttgcacgtatgctgaatttgggtcaccgtcgccatcttgagcgtaacctgctgctgagcgccctctatcagacccaaagttatcttcaccatctgcaggagattaaccagcgcgatgggctgaacaaacccgatgccgcactgctaaaagagatggatcgcctgctactggtggctatccagacctcctcgcccaaagctgccgtacagcaacttactgaggtgatgcaggcattgcctgcccatgctgactcgccgctggtggaggaaatcttgcaagagttcagcgccagcctgtatcagctgctgccgttgtccatcacgcttgaaaatagcgatctgagcattacctggtacatgtaccacgtcaaagcgttggtggcgtttctcaatcagggcatcaatatctatgtacaaaaggtgggggaggaatcgctgcagcgtagccaacaaaaccacaaagccttgcaatcgatcatcacgtctattggtctgtttgccctgttggcactggttatcaccgggtttgccggctggtatatctaccataaccttggctctaacttaacggcgatatctcatgccatgacccgattggcaagaggagaaaaagaggtcagcgtaccagcccaacaacggcgtgatgaactgggcgaactggctcgcgcgtttaacgtttttgcccgcaataccgcttcgctggagcagacatcacgtcttctgaaagagaaaagtacgctattggaaaccacctttcacgctatgcgcgatggttttgccctgttcgacaacgagggctttctggtggtgtggaacccgcaatacccactgttgctggggctggcaccggagcagctacagcatggtcagcactaccttcaattattgaagcagatgacgccactgcaagagcatatacttgagaacctcgccctcccgctgccaaaaacccaagagctaagacttgaggaccatcgcactatcgaactgcgtttcagtccggttcctggacgagggatggttaatgtggtgttggatcgtagcgagcgcaaagcactggaagaagcgctggtccatagccaaaaaatgaaggcggtagggcagctcacgggcggcctggctcatgattttaataacctgctggcggtgattattggcagtcttgagctaaccgctacggactcgtcggatgccacgcgtattcatcgtgctctgaaggccgctgagcggggggcgcaactcacccaacggttgctggcgttctcacgcaagcagtcgcttcaccctcgagctgttgcgatgaaagaactacttgataacctggacccgctgatacgccactcgcttccggctcatcttacgctcacaattgaagctcagcagcctgcctggcacgcctggatagacgtaaaccaactggaaaacgcaattatcaatctggtgatgaatgctcgcgacgcgatggaagggcgcagcggcgagattaaaatccgcacctggaatcaacgcgtagagcgtggtgaagggcgcaaacaagatatggtggtgctggaagtggctgatagcggccatggcatgaccaccgcagtgaaagagcaggtttttgaacccttcttcaccaccaagcaaaccggtagcgggagtgggcttgggctgtcaatggtatacggctttgtgcgccagtccggagggcgggtacagatagaaagtgaaccggggaaagggacccgggtctgcttgcagttaccccgtgcactcacacaaagtctgatagaagtcctgccagcgcttggtgccgttgcgaatatggctgatcagctagtcttagtgctggaagatgagccggatgtacgccagaccctgtgcgagcaactccatcaactgggctacctgacgcttgaaaccggcgacagtcggcaggcgctggcattgatggccgacgtgccggatatcagcattgtgataagcgacttaatgctacccggcgacatgaccggtgcggaagtgcttcagcaagcgcgcagtgtttatcctcatcttaagctgttgttaattagtggccaggatctgcggcgcagcaagaatttcatgccggaggtggaactgctgcgtaagccttttaaccaacaacagctagtacaggcgctgcaaagagtctga

SEQ ID NO: 43 - protein, 851 aa, Yersinia pestis
MEQPRLSFFASVRGRLLFFNLLVVAVTLMVSGVAVLGFEQASRLQKQVQERTLRDMSSSMALARDTANVATAAVRLSQVVGALEFQSEAASLQETQLALRSSLAHLANAPLASHEPLLVKRIIERSNELETSVARMLNLGHRRHLERNLLLSALYQTQSYLHHLQEINQRDGLNKPDAALLKEMDRLLLVAIQTSSPKAAVQQLTEVMQALPAHADSPLVEEILQEFSASLYQLLPLSITLENSDLSITWYMYHVKALVAFLNQGINIYVQKVGEESLQRSQQNHKALQSIITSIGLFALLALVITGFAGWYIYHNLGSNLTAISHAMTRLARGEKEVSVPAQQRRDELGELARAFNVFARNTASLEQTSRLLKEKSTLLETTFHAMRDGFALFDNEGFLVVWNPQYPLLLGLAPEQLQHGQHYLQLLKQMTPLQEHILENLALPLPKTQELRLEDHRTIELRFSPVPGRGMVNVVLDRSERKALEEALVHSQKMKAVGQLTGGLAHDFNNLLAVIIGSLELTATDSSDATRIHRALKAAERGAQLTQRLLAFSRKQSLHPRAVAMKELLDNLDPLIRHSLPAHLTLTIEAQQPAWHAWIDVNQLENAIINLVMNARDAMEGRSGEIKIRTWNQRVERGEGRKQDMVVLEVADSGHGMTTAVKEQVFEPFFTTKQTGSGSGLGLSMVYGFVRQSGGRVQIESEPGKGTRVCLQLPRALTQSLIEVLPALGAVANMADQLVLVLEDEPDVRQTLCEQLHQLGYLTLETGDSRQALALMADVPDISIVISDLMLPGDMTGAEVLQQARSVYPHLKLLLISGQDLRRSKNFMPEVELLRKPFNQQQLVQALQRV

SEQ ID NO: 44 - DNA, 1764 bp, Yersinia pestis
  CDS (1)..(1764)
atggacgacttaaccctgcgttattttgacgctgaaatgcgctacctgcgcgaagcgggcgaagagtttgctcgtgcgcaccctgatcgggctgccggattaaatctggataaagccggggcgcgtgacccctatgtcgaacgcctatttgaaggctttgcctttctgatgggccgactgcgggaaaaactggatgacgacctgccggaactgaccgaagggttggtcagcctgctgtggccgcactacctgcgtaccattccgtcgctttctattgtggagttcaccccacaatggcaggggatgaaagagcgcatgggggtgagtaaaggttttgaagtgctgtcccgccccattggtgaacgtgaaacccgttgccgctataccaccacgcaagagatggacctgctcccgctgaccctgcgccgtgccgggctggacagtgaacccgatggccggtcggttatccggctgcgctttgattgcagcgcgctggccgactggagccgtatcaacctgagccgcctgccgctctattttgatgccgatgcgccgctggcctgcgccctgcatgaggcgctgaccctgaataccgccaaactctggatccgtttgccggggcaggctgatcgccagccgctggacggccatttcgcgccgctggggttcggggagcaggatacactgtggccgaaggccgacagtgcgttcagtggttatcagctcttgctggagtactttaccttccgcgaaaagtttatgtttgtcgcgttgcaggggctggatgggatcgagctgccggcggaactgccgtggtttgagattgatgtggtgctggaaaaacgctggcaacatgatttctcgttctccgaaaagaacctgcgcctgcattgtgtgccggtgattaacctgttcccgctggaatcggacccgctgtcactttcttcattacagaccgaatacctgctgcgcccaatgcgcattcaggacggttataccgaagtgtattcggtggactcggtgatctcctcccgtcataccgggcatcaggtctatgtccctttcaccagctttcgccacaagggggggatgttgcgccatgatgcgccggagtattactaccatacgcgggtcaaacgcgggccttcggggttacacgatacctggctggtgctggggggcgaggcgtttgataaccacagcgtaccggataatgaaaatttgtcgctgagcctgaccggcaccaacgggcagttaccgcgtaaagcgctgcaaagcacggtgctggatacggcggtgaaatccaccggggctcaggtgcgggtgcgtaatctctccgcgccgtcattgccatgctacccgccgaaccgcgaccgttttcactggcgggtgctgagccatctgggcagtagttttctgtggatgatggataacgccgaagtgctgcgcggcacgctggcgctgtatgactggacggacaacgaaatgaaccgccgtcggctggaggcgatcgccgaggtcaaacacagcgagattgaacgctttgagcggggctatttgctgcgcggggtgcacattgaaatcaccctcgacagcaacggctttaccggtaccggggatatttgtctgtttggggaaatgctcagtcgcttctttgccctgtataccgatattcacctgtttaatcgtctgactctgatactgcaaccgacaggagaacgtttggaatgggaagagaatcatcagtcccgccttcccggctaa

SEQ ID NO: 45 - protein, 587 aa, Yersinia pestis
MDDLTLRYFDAEMRYLREAGEEFARAHPDRAAGLNLDKAGARDPYVERLFEGFAFLMGRLREKLDDDLPELTEGLVSLLWPHYLRTIPSLSIVEFTPQWQGMKERMGVSKGFEVLSRPIGERETRCRYTTTQEMDLLPLTLRRAGLDSEPDGRSVIRLRFDCSALADWSRINLSRLPLYFDADAPLACALHEALTLNTAKLWIRLPGQADRQPLDGHFAPLGFGEQDTLWPKADSAFSGYQLLLEYFTFREKFMFVALQGLDGIELPAELPWFEIDVVLEKRWQHDFSFSEKNLRLHCVPVINLFPLESDPLSLSSLQTEYLLRPMRIQDGYTEVYSVDSVISSRHTGHQVYVPFTSFRHKGGMLRHDAPEYYYHTRVKRGPSGLHDTWLVLGGEAFDNHSVPDNENLSLSLTGTNGQLPRKALQSTVLDTAVKSTGAQVRVRNLSAPSLPCYPPNRDRFHWRVLSHLGSSFLWMMDNAEVLRGTLALYDWTDNEMNRRRLEAIAEVKHSEIERFERGYLLRGVHIEITLDSNGFTGTGDICLFGEMLSRFFALYTDIHLFNRLTLILQPTGERLEWEENHQSRLPG

SEQ ID NO: 46 - DNA, 669 bp, Yersinia pestis
  CDS (1)..(669)
atgttgtcaggtattcaatcggcatcactaatatgtcattcgtcttcaatagaagaaagtgaaaaaattcaatcagttgtttttttatctgatatcggtcttgataaaaacaggttaacactagagcaagaaaagggattgaaggatataaaatcatctatagatgattgttatcacaacggtattagtactagtactggcaggaaaaaaataaaagaattaagaaataaagttacccagtacattaactctgtagagagttaccgggataagatttatgatgttatcattgataaaagaacgggcagaggtgaaaagataatattaaaatctggatgtgatgaaacacatagaaatagttatcttaaggggattatttatctcagtaaattgcaggatcttattcgtaatgaaatagataatgcacctattaaatataaaaaatcactaagtaaagtttttgatgtcatgggaaaaggcgctgtgtatggcgacataaaggcactgaatgaagatcgtaaattgcctgattttaaatatagtgactctgaatgttctgcgtatgattatagctatggtaaccatgcacttgaatgtggtattcgctctttggaatgcgcaggacagactggattactgatctgtatgtgtctaatgggagggaaatga

SEQ ID NO: 47 - protein, 222 aa, Yersinia pestis
MLSGIQSASLICHSSSIEESEKIQSVVFLSDIGLDKNRLTLEQEKGLKDIKSSIDDCYHNGISTSTGRKKIKELRNKVTQYINSVESYRDKIYDVIIDKRTGRGEKIILKSGCDETHRNSYLKGIIYLSKLQDLIRNEIDNAPIKYKKSLSKVFDVMGKGAVYGDIKALNEDRKLPDFKYSDSECSAYDYSYGNHALECGIRSLECAGQTGLLICMCLMGGK

SEQ ID NO: 48 - DNA, 1602 bp, Yersinia pestis
  CDS (1)..(1602)
atggcgacgttaactaccttgctcaccgcttgtgccgcagagcctgagccacttttacagcaggccagacaacaggttgcgctctgggaacgttggttgcaaccggtgacacccgataagcataccggtgaagaccccggttacgatgaccacttccaacagatgcgtgaagaggtcaataagctctcgggtgccgataccacgctgacctgcgagctggctgaaaagctgttcaccacacacggcaaggatgtgcgggtggcaacctattacgtctgggcgcggttgcaccgtgacggcgaggccgggctggcggatggcctttccctgctggccgggctgatcacgcgctttggtgagggcctgcatccgctgcgtacaaccagccggaaaaccgcgttggagtggctggccggctcccggatgcgcgacagcttgtcactttacccggaagtggataaagccgattttgagcgtatcgtgggcgcactggcccttatcgaacagaggctcagtctctgggacgaaggcgtccggccgcaactgggcgggttgtataccgccctggaaaaccgcctcgcgcaatctggtgggctaaatgccgtggtgccgcaaaacagcagcgggtcatcatcggcaggcagcttgaacccagccaacacggcgtcaccggcgttgcggccagtgcaatcgggacgtgacctgctcgatcagaccaaaacgctggcaaaatacctgcgcaaccagccgcagggctggctctccgggcatcatcttatcaaaagcgtgcgctgggatacggtacatcagtcaccgccattagatgtgaacggacggacccggctggtaccgccgcgcccggaatatcgcgtacaactcaagcgcctttatctgcaacaaaactggctggcgttgctggaacaggccgaatcgatatttgccgaaggggttaaccatttctggctcgatgtgcagtggtatctgcatcaggccctcagcaaagcgggggccccattcgatggctgggcgagctgtatcacgcaggatttacggttattactgacccggctgccggggctggaagggctatgctggagtgacggcacgccatttgccgatgaggtgacgctggggtggataaaccagcaggtactggagtcggtctcgggctggggcagcgagccggccgccgtttcatccggtgaggatggcattctgttgctggagccagaagctttggctcaggcggacagcgaaggaattgaagcggcgcttaactggttgcagtcacgaccggggatcaccacggcgcgccatcagtggctgctgcgtctggtgatggcccgggtggcggagcagtacggtaagaacgatatggccttgcatctgctgagtggccttgatagcagcggcgcgttgttgaccttgccacagtgggagccggggttggtgtttgaagttaaagcccgccgtttgaagctgttgcgtatgaaggcacagcggggtgacagtgataaaacccgcctgcatgctgaaatggaaagcctgctcagtgggcttatcgccctcgacccggcccgtgcggccgtattatgcggataa

SEQ ID NO: 49 - protein, 533 aa, Yersinia pestis
MATLTTLLTACAAEPEPLLQQARQQVALWERWLQPVTPDKHTGEDPGYDDHFQQMREEVNKLSGADTTLTCELAEKLFTTHGKDVRVATYYVWARLHRDGEAGLADGLSLLAGLITRFGEGLHPLRTTSRKTALEWLAGSRMRDSLSLYPEVDKADFERIVGALALIEQRLSLWDEGVRPQLGGLYTALENRLAQSGGLNAVVPQNSSGSSSAGSLNPANTASPALRPVQSGRDLLDQTKTLAKYLRNQPQGWLSGHHLIKSVRWDTVHQSPPLDVNGRTRLVPPRPEYRVQLKRLYLQQNWLALLEQAESIFAEGVNHFWLDVQWYLHQALSKAGAPFDGWASCITQDLRLLLTRLPGLEGLCWSDGTPFADEVTLGWINQQVLESVSGWGSEPAAVSSGEDGILLLEPEALAQADSEGIEAALNWLQSRPGITTARHQWLLRLVMARVAEQYGKNDMALHLLSGLDSSGALLTLPQWEPGLVFEVKARRLKLLRMKAQRGDSDKTRLHAEMESLLSGLIALDPARAAVLCG

SEQ ID NO: 50 - DNA, 2448 bp, Yersinia pestis
  CDS (1)..(2448)
atgattatcagattccggctaccgccattatcggtactgatatatggctcaatattgttaccgtcactgatacccttgactattttaggagggagcctccctccgccctcgacatcaacaacgatgcctgatactacgttatatcttgaactcgtggtcaatgaccgtaactttgggagtactgtacctattagttaccgcaacaaccgttattacctttctcaatcacagctaagaacgattgggcttcctatatcagagccactggcacccgagattgccattgataatatggcgggcgtgaacgttaaatatgatggagagaaccaacgcctattaatcaatgttcccagcgagtggctaccaaagcaacaaatcgaggttaccgaacaagatgatttcaatcttgcgcaaagtagtttgggggctttattcaattacgatatctatgccacacaaggttacccgtactcatctttaactcatttttcagcatggacagaacagcgcatttttgatcgttttggcttgctcagtaataccggcgtttatcgcacccacttcccaagtaataacaacacagatgacgcgaaaggctatattcgttttgatacccagtggcagaagaatgacgaagaacatctgctgcgttatagcacaggtgacctcattaccggtgctttgccatggagcagtgcgatacgtcttggcggcattcagatagcacggcattttgctattcggcccgatctcataacctacccattaccacaattttctggtcaggccgcagtccccagcaccgttgatctctacattgataacttcaggacacaatcggccaatatcaacccaggcccttttgtcatcaataatgcaccgcgaatcaatggtgcaggccaagcgactatcgtcacaaccgatgcccttggcaggcaaatcagcacatctgtacccttttacgttgccagtacactgctaaaacccggagtgtgggatttcagtctctcggggggtgccctacgtcgtaattatgcgattcgctcagcagattacggtgaaatggttgccagtggtgtcgttcgttacggcacaacaccttggttgacactggagggccggggagacatcgccaaagaaatgcatgtaattgggggtggcgttaattttcgcatggggttattgggtgtcctaaatagtgcatacagtataagcaacacatcgaacggcgcttttaacaatgtggctgaaccgctaaataccaataatgccacgccgaatagattaccgtccccagcagcgtcacgccgtggacgtggcaaccagcgttcattaggctatagctacagtaatgcattctttaatttaaatgctcaacatattatctctagtgatgaatacagtgaccttgccaattataaaacgccttctctcttaagccgacgcatgactcaactcacgggaagtctcagcctcggtagttacggcaccgtgggatcgggttactttgatgtcagggatgctcttggcgaacaaacccggctcattaatatcagctacagcacctcactgttacgcaatagcaacttttatagcgcactaaaccgtgagctgggccgtaaaggttacaatgtacaactggtctggagcattccgttaggcccgcggggatcaagcagcatcagtgcaacccgcacaaatgataaccaatggattcaacaactgaattatagccgttcagcgccctcgaatgggggattgggctggaacctcgcttatgccaacagcacgaataacaataatcaataccaacaggcagacattgtttggcgtacttcaatgatggagagtcgcatgggcctatatggtaacagtaataactacaactattgggggggattaaccggttcattagtggtgatgaataggagtgtctatgccagcaatatgattaacgatgcttttgctttggtatcgaccaatggttttagtaatattccggttagttacgaaaatcaacttattggcacaactaatgccaagggttatttgctgatcccgacagtcgcctcatattatcaggcaaaattccagattgacccaatgaatttaccggcagatgtcatgctacccaatgttgaacggcggttagccattggtgaacgcagtggctatttaattaatttccccattaagcgaatttccgcagtgaatattagaattacagacgcatcaggccaagacttaccgaaaggcagtgcaatatatactaccggtaacatccccataagttatgtcggttgggatggcatggtatatatcgagcaagtggcacaattaaataaccttaggattatacgtgccgataatggcacacagtgctacagccagttcaaactaaagacgacggaaggtatacaggatgctggcaccacagtatgccgatag

SEQ ID NO: 51 - protein, 815 aa, Yersinia pestis
MIIRFRLPPLSVLIYGSILLPSLIPLTILGGSLPPPSTSTTMPDTTLYLELVVNDRNFGSTVPISYRNNRYYLSQSQLRTIGLPISEPLAPEIAIDNMAGVNVKYDGENQRLLINVPSEWLPKQQIEVTEQDDFNLAQSSLGALFNYDIYATQGYPYSSLTHFSAWTEQRIFDRFGLLSNTGVYRTHFPSNNNTDDAKGYIRFDTQWQKNDEEHLLRYSTGDLITGALPWSSAIRLGGIQIARHFAIRPDLITYPLPQFSGQAAVPSTVDLYIDNFRTQSANINPGPFVINNAPRINGAGQATIVTTDALGRQISTSVPFYVASTLLKPGVWDFSLSGGALRRNYAIRSADYGEMVASGVVRYGTTPWLTLEGRGDIAKEMHVIGGGVNFRMGLLGVLNSAYSISNTSNGAFNNVAEPLNTNNATPNRLPSPAASRRGRGNQRSLGYSYSNAFFNLNAQHIISSDEYSDLANYKTPSLLSRRMTQLTGSLSLGSYGTVGSGYFDVRDALGEQTRLINISYSTSLLRNSNFYSALNRELGRKGYNVQLVWSIPLGPRGSSSISATRTNDNQWIQQLNYSRSAPSNGGLGWNLAYANSTNNNNQYQQADIVWRTSMMESRMGLYGNSNNYNYWGGLTGSLVVMNRSVYASNMINDAFALVSTNGFSNIPVSYENQLIGTTNAKGYLLIPTVASYYQAKFQIDPMNLPADVMLPNVERRLAIGERSGYLINFPIKRISAVNIRITDASGQDLPKGSAIYTTGNIPISYVGWDGMVYIEQVAQLNNLRIIRADNGTQCYSQFKLKTTEGIQDAGTTVCR

SEQ ID NO: 52 - DNA, 1233 bp, Yersinia pestis
  CDS (1)..(1233)
atggaaatagttaattttattagtgctcaggatatagttgagattgaatttctctcaacagagaatgaaaaaaataaagaggcattgaattcagtaaataaatgggagaatgatgctccatttggggaaaacagaaccaatgctgctaatgaaattcgtgatgttatagagagaaatgcgcctatacttaggctatcgcgtctgaatatcagttctttacctgatgttttaccgcatagtctcatcgagattgaaatttattattgcgatgaattatctactctccctgacagttttccttccgagcttactaagctaaaaattagccattgccctgaaatttcatctttatataagaatgcaccaaagcgtttgactaaattagaaataatatcttgtccgaaaatttcaaatgctataattccactgcctgaatcattacaatatattaaattagatatagactcgaaagagcgcctttctctctcgtttgataaatttcctaaaaatttaagaggtattaatttaagtgatagttttttaattgaaaaaagcaaattcaaggatagagagatccgattgaatgttctagtgccatccgtagctctagagtttaaattaggggacattctatatggaattgctcagtgtcaacatgaggtgatgcagcaactaattaattttaatgacttttctaacaaagatatttgttcgcaaacaacaattactgatgctgtttgggaacatcgtaattatttttcccgtgataaatatagagacgatgcgacaatcaaagaaatgttaaatgatgctgatcgcggaattaaattcaaagatttcctagaaaaacatgaaaagtataatattttaagtcgttcgggaataaaatcataccgtcctcataaaaatgaggaggatatatgtttatcaaggaccagtaaggctggtcttgaatttcagatcatggaaaggcaagagcgtgtatttttttgtatagataatttgaataattgtattcctgaaattgcacaaaaaaaacctgattatggtacctatatcacagcctctgagttgagatggctttaccgtcgtaaagatcaccctaatgtaaaaaataatgttcaattctgcttggagggagcatttatttcacaagaagaggtattttcactgcctggttgggaaacttattttcctaaaagaaaaagtaacttcataccttcctatgtttga

SEQ ID NO: 53 - protein, 410 aa, Yersinia pestis
MEIVNFISAQDIVEIEFLSTENEKNKEALNSVNKWENDAPFGENRTNAANEIRDVIERNAPILRLSRLNISSLPDVLPHSLIEIEIYYCDELSTLPDSFPSELTKLKISHCPEISSLYKNAPKRLTKLEIISCPKISNAIIPLPESLQYIKLDIDSKERLSLSFDKFPKNLRGINLSDSFLIEKSKFKDREIRLNVLVPSVALEFKLGDILYGIAQCQHEVMQQLINFNDFSNKDICSQTTITDAVWEHRNYFSRDKYRDDATIKEMLNDADRGIKFKDFLEKHEKYNILSRSGIKSYRPHKNEEDICLSRTSKAGLEFQIMERQERVFFCIDNLNNCIPEIAQKKPDYGTYITASELRWLYRRKDHPNVKNNVQFCLEGAFISQEEVFSLPGWETYFPKRKSNFIPSYV

SEQ ID NO: 54 - DNA, 2082 bp, Yersinia pestis
  CDS (1)..(2082)
atgaaaggccgcctactggacgccgtacccctcagtacactttccggagttggcgcaagtcaggctggaaaactcgccaagataggtttggaaaccattcaggacctgctgctgcaccttccgctacgttatgaggatcgtactcgtctatatcgtattggtgatctgctgcccggtctttcggtcacggttgaaggtgaagttctacgctctgatattagctttggtcgccgccgtatgatgacctgtcaaatcagcgatggtagcggcgttctcaccctgcgcttttttaacttcaatgcagcaatgaaaaacagtctgtcagtaggcaaacatgtcattgcttatggcgaagccaaacgaggcaataacgggccagaaatcattcaccctgaatatcgggttcatggcgagaatattggcgttgaattacaggaatcgctgacaccagtctatcccacgaccgagggcattcgtcaggcaactctgcgcaaacttatcgatcaggcactggcaatgctggacacctgtgttattgctgagttactgccaattgaattgagccgttctctcatcagcctgcctgaagcgattcatactctgcatcgccctccggcagatattcaattagctgatttagaacaagggaagcaccccgctcaacggcgtctgatcatggaagagttgctggctcataacctcagtatgttggcagtcagggcgggtgcacaaagctaccgagcattgccattaatggccgaagagcagcttaaacaacgttttctggccgccctaccttttacgcctactcaagcccagcaacgggtcgtcgccgaaattgaacgggatatgacccaaagtttcccgatgatgcgactgattcagggggatgtaggttccggtaaaacactggtagccgcactggccgcgctacgggccatcgctcacggtaagcaggttgccctgatggcaccgaccgaactgctggcagaacagcacgccactactttccgtcaatggctggagcctcttgggtttagtgtcggctggttggctggcaagcaaaaggggaaagcgcggttagcacaacaagaggccgttgccagcggtcaggtctccatggtgatcggtactcatgccatgtttcaagaacaggtgcaattctcggggttagcactggtaattattgatgagcagcatcgtttcggtgttcaccagcgcctcacgttatgggaaaaaggtgaggagcaaggcttccatccacatcaattaatcatgacggcaaccccgatcccaagaacattagccatgactgcctatgcagatctcgatacctccgtgattgatgaattaccgccaggcagaacccccgtgacaacggtggcaatcccggatactcgccgtagtgatgttattcagcgggttaaaaacgcgtgcctggaagagggtaggcaagcatattgggtatgtaccttgattgaagaatcagagttgctggaggctcaggctgcagaggtaacgtgcgaagagctgaaaattgccttaccggaaattaaagttggcttggttcatgggcggatgaaaggccccgaaaaacaggcgattatgctggcctttaaacaaggtgagctacagttattggtcgcaaccacggtgattgaagtgggagttgatgtgcccaatgccagcttgatgattatcgataatcctgaacggttggggctggcgcagttacatcagttacgtgggcgtgtcgggcgcggtgctgtcgcttcccactgcgtattactctataaaacgccattaagtaaaaccgctcaaatgcggttgcaggtattacgtgatagcaatgatggttttgtgattgcacagcgagatctagaaatccgtggccccggtgagctattgggcacacggcaaacaggtagcgcagagtttaaggttgccgacctacttcgtgatcaagcgatgatcccagaagttcagcgggtcgcccgtcatctgcatcaacagtaccctgaacatgccaaggcattgatagaacgctggttgccggagcgggtacggtataccaatgcgtaa

SEQ ID NO: 55 - protein, 693 aa, Yersinia pestis
MKGRLLDAVPLSTLSGVGASQAGKLAKIGLETIQDLLLHLPLRYEDRTRLYRIGDLLPGLSVTVEGEVLRSDISFGRRRMMTCQISDGSGVLTLRFFNFNAAMKNSLSVGKHVIAYGEAKRGNNGPEIIHPEYRVHGENIGVELQESLTPVYPTTEGIRQATLRKLIDQALAMLDTCVIAELLPIELSRSLISLPEAIHTLHRPPADIQLADLEQGKHPAQRRLIMEELLAHNLSMLAVRAGAQSYRALPLMAEEQLKQRFLAALPFTPTQAQQRVVAEIERDMTQSFPMMRLIQGDVGSGKTLVAALAALRAIAHGKQVALMAPTELLAEQHATTFRQWLEPLGFSVGWLAGKQKGKARLAQQEAVASGQVSMVIGTHAMFQEQVQFSGLALVIIDEQHRFGVHQRLTLWEKGEEQGFHPHQLIMTATPIPRTLAMTAYADLDTSVIDELPPGRTPVTTVAIPDTRRSDVIQRVKNACLEEGRQAYWVCTLIEESELLEAQAAEVTCEELKIALPEIKVGLVHGRMKGPEKQAIMLAFKQGELQLLVATTVIEVGVDVPNASLMIIDNPERLGLAQLHQLRGRVGRGAVASHCVLLYKTPLSKTAQMRLQVLRDSNDGFVIAQRDLEIRGPGELLGTRQTGSAEFKVADLLRDQAMIPEVQRVARHLHQQYPEHAKALIERWLPERVRYTNA

SEQ ID NO: 56 - DNA, 1220 bp, Yersinia pestis
atgaacagtgaaaaaaaagaacatcttaatacagtatcaagaaagttaagaacgatcatggagcgtgataatattgatgctgtcattgtgacaacgtgtgataatttttatcatgttaccggtattctcagtttttttatgtatacctttcgaatacgggcacggctatcgccgttgtcttccgtgatgtgaaaattccatcgctaatcattatgaatgaatttgaagcagccagcttaacgttggatatgccaaatgctgaattaaagacctttcctgtttgggtagatgtagatgatccgtttaacatgcgcgattctgcaaataataataaagagaggccgataggtcctcctattgagagtgtatgtaatatactcaaagacgcattgaacgatgccagggtacttaataaaaaaatagcaatagacctcaatataatgtcaaacggtggtaaaagagttattgatgccgttatgcctaatgttgattttgtcgactcaagctctatattcaacgagctaagagtgataaaaagcccttgggaaattaaaagactcagaaaaagtgcagaaattacggaatatgggatcactgaagcatcaaagctcatccgggtaggatgcacatcagctgaactcactgccgcatataaggcagcagtaatgagtaaatctgaaactcacttttcaagattccatcttatctctgttggcgctgatttttcacccaaactcataccaagcaatactaaagcctgttcaggcgacttaattaaatttgattgcggcgtcgatgttgatggttatggtgccgatattgccaggacttttgttgttggggaacctcccgagatcacccggaagatataccaaacaataagaactgggcatgaacatatgctgagtatggttgcgcctggcgtcaaaatgaaagatgtatttgattcaacgatggaggtcattaaaaagtccgggttacctaattataacagagggcatcttgggcatggtaatggcgtctttttgggattagaagagagtcccttcgttagcactcatgctacagaatcgttcacttcaggtatggtgctaagtttagaaacaccttactacggctataatttgggttcgattatgattgaagatatgattttaataaataaagagggtattgagtttttgagtaaattacccagggatttagtttcttttaattaa

SEQ ID NO: 57 - DNA, 965 bp, Yersinia pestis
atggataagaaacgcgtatttattgccgggcatcgtggcatggtgggttctgctattgtacgtcaacttgaaaaccgtaatgatattgagttgatcattagggatcgtactgaacttgacctcatgtctcaatccgctgtgcaaaaattttttgctactgaaaaaattgatgaaatctatttggctgcggcaaaagtggggggattcaggccaataataattatccggcagagttcatctaccaaaacttaatgatcgagtgcaatattattcacgcggctcatttagctggcattcaaaaattattatttttggggtcttcttgtatttatccaaaattggctgcacaaccaatgacagaggaggctctgttaactggcgtcttggaaccaacgaatgaaccttatgccatcgccaagatagccggtatcaaactgtgcgaatcttataatcgtcaatatggtcgcgattatcgcagtgttatgccaaccaacctttatggtgaaaatgacaattttcaccccgaaaattcccatgtcattcctgccttattacgtcgcttccatgaggctaaaattcgtaatgataaggaaatggttgtgtggggaacgggtaaaccaatgcgtgagttcctgcatgtagatgatatggctgctgccagtgtgcatgtcatggagctgtctgatcaaatttatcaaaccaatactcaaccaatgctttcgcatattaatgtcggaacgggtgtggattgcactattcgtgaattggcagaaactatggctaaagttgttggtttcaccggaaatttagtttttgattcaactaagccggacggaacaccacgaaaattgatggacgtaagccgcttggctaaactcgggtggtgttatcagatttcgcttgaagtaggtttaacgatgacttatcaatggttcttggctcatcagaataacttcagaaaatag

SEQ ID NO: 58 - DNA, 1076 bp, Yersinia pestis
atgattaataatagtttctggcaaggtaaacgggtttttgtaacaggccatactgggtttaaaggtggctggttgagtttatggttgcaaaccatgggggcaacggtaaaaggttactctttgcccccccccccacggtgcctagcctatttgagaccgcacgagttgccgacgggatgcaatcggaaatcggtgatattcgtgatcaaaacaaattattagaatcaatccgcgaattccaaccagagattgttttccacatggctgctcagccactggtccgtctatcctattccgagcctgttgaaacctactcgacgaatgttatgggtaccgtttatttactggaagctattcgccatgttggtggcgtcaaagcggtggtcaatatcaccagtgataaatgctacgataataaagagtggatctggggctatcgcgaaaatgaagcgatgggggggtatgatccttactccaacagtaaaggttgtgcggaattagtgacgtcatcctaccgtaattcgttcttcaatccagcgaactatggccagcatggcactgccgtagcgacagtgcgtgcgggtaatgttatcggtggtggcgattgggcattggatcgcatcgttccagatattcttcgggcgtttgaacagtcccaaccagtgattattcgcaacccacatgccattcgcccatggcagcatgtgttggagcctttgtcgggttatttgctgttggcacagaagttatatactgacggtgctgaatatgccgaaggttggaactttggtcctaacgatgctgatgctactccagtaaaaaacattgttgaacaaatggtgaaatattggggagagggtgcaagctggcaattagatggcaatgctcaccctcatgaagctcattatctgaaactggattgttcaaaagctaaaatgcaacttggctggcatcctcgctggaacttgaatactacgctcgaatatattgtgggctggcacaagaactggttatcaggcacagatatgcatgaatacagtattactgaaattaataattacatgaacactaaatga

SEQ ID NO: 59 - DNA, 220 bp, Yersinia pseudotuberculosis
gtgctgaagcaacgcagagaagacaaagaatacaataccaaaaatcttcattaaaacgctatatgttattatctcactaggttttaaaacttgggacattactatgtaatcagtctgaataataattgtagttaaaatagaaaaaattagaaaaccagaggcacgacggacaataggcttaataatatcagatctaattctacgaagcttgcatgcctgc

SEQ ID NO: 60 - DNA, 265 bp, Yersinia pseudotuberculosis
ttcgccgagggatatcactccgcaaataccaccggcatagagacaatctcgccacgccttgccgtcttttagcgttacgagagcggccaggcagatcgaagcgccgacaccgtaaatagtgtgcccgtcacaccagcgggataaataatttcagcaagatcgctatcaacataatcccaacccctgcgccaaatggcttacaggatttgtgggggcaagcgtggtttctggataaaggccagcggctaggccgaattcgagctcg

SEQ ID NO: 61 - DNA, 571 bp, Yersinia pseudotuberculosis
tcaataaggtgtacgcgcctgagttggaaattgaagtaaaaaactaacacaacggttacgggcaattgacaataattctattgaacaatttcttattctgcgtcgtcattatctgccgcatgaaaatgacgatatagaaagtctggcccgtgccgtttggttagataaccgttattgggataatacccgtatttctattgccaatggaattggtctggcatttaaaggcgattaatgaaaagtctcgattttaccttaagcatgattgataaaatcacacggccattaaaatccgtgcaagcctctgtaaaaggatttgcggaaagttctcaggcggctttcggtaaaattgccattggcggggccgcgctgttcggtgtcgtacagggaatgaagggcgcactgggacccgcagctgaatttgccggcgcgcttaatgaggccagcgccaagggcgtgagtgattgcgccttgcaaaagatgagcgctgacgccctgaaattcagtatgcaatatggccgcagcgccgtgggatgttgtgcgttcaaatgcagacatggcgcaagtgcaa

SEQ ID NO: 62 - DNA, 423 bp, Yersinia pseudotuberculosis
aagccacagttatcttgatgtggacattatgcattgtaatagcccattaacatgcgctaagaagcataaattaatatacagtaacaagcattacgaggtgctaatgtttggaaagcagcctcttccaactacaataataagttaactatcctccacccacaaataattacaaatgacccctttccgctataactccgatctcaccagcggctcgctacaaacccgagagtgtcgcattattactggcttgttactccaagagcttgatgaagctgcctgggataaagccatgtataaggaaaatgtgcttcagaaacgcacgcaatctacggtaagacgtatttcttcagcgcttagaaaacgcctagaacacctaagctctgacttttgggcttttgcgtttttatgctaggccgaattcga

SEQ ID NO: 63 - DNA, 380 bp, Yersinia pseudotuberculosis
gctgagtagcctgatatttttatggtgaaaaagataaacaatcatagaggggataaatatgagccagttctaccattgttcagataagtttaagcaagatactgtcaatcacagtgaccattcatggtcattacacttcctccagttgccagacaatgcctaacataaaaacgcaaaagcccaaaagtcagagcttaggtgttctaggcctaatccaagctcggaagaaatacgtcttaccgtaaattgcgtgcgtttctgaagcacattttccttatacatggctttatcccaggcagcttcatcaagctcttggagtaacaagccagtaataatgcgacactctcgggtttgtagcgagccgctggtgagatcggagt

SEQ ID NO: 64 - DNA, 243 bp, Yersinia pestis
  CDS (1)..(243)
atggaaacaagattcctacaggcaaacaaagaagcacgctgggccttcggcttaacactggcctacctcgcagggtggattataaccgcttacttaccgggcaaccttcccggaatgagcggcctacctgcttggtttgaagcggcctgtatcgcattaccgctcctgtttattgtgctgtgtattctgatggtgcggctgatattccgcgatattccactggaggatgacgatgcaaactga

SEQ ID NO: 65 - protein, 80 aa, Yersinia pestis
METRFLQANKEARWAFGLTLAYLAGWIITAYLPGNLPGMSGLPAWFEAACIALPLLFIVLCILMVRLIFRDIPLEDDDAN

(19) United States
(12) Patent Application Publication
(10) Pub. No.: US 2005/0079168 A1
(43) Pub. Date: Apr. 14, 2005

(54) Virulence genes proteins and their use

(76) Inventors: Audrey Vladimirovich Karlyshey, London (GB); Brendan William Wren, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/496,207

(22) PCT Filed: Nov. 18, 2002
(86) PCT No.: PCT/GB02/05212

(30) Foreign Application Priority Data
Nov. 19, 2001 (GB) 01276575

(51) Int. Cl. A61K 39/395; C07K 1/00

(52) U.S. Cl. 424/130.1; 530/350

(57) ABSTRACT

A series of genes from Yersinia species, in particular, Yersinia pestis and Yersinia pseudotuberculosis, are shown to encode products which are implicated in virulence. The identification of those genes therefore allow attenuated microorganisms, vaccines, and other therapeutic products to be produced.

### FIELD OF THE INVENTION

This invention relates to virulence genes and proteins, and their use. More particularly, it relates to genes and proteins/peptides obtained from Yersinia pseudotuberculosis, and their use in therapy and in screening for drugs.

### BACKGROUND OF THE INVENTION

Yersinia pseudotuberculosis is an organism that is implicated in gastroenteritis, terminal ileitis and mesenteric adenitis in humans and Yersiniosis in livestock. It is desirable to provide a means for treating or preventing conditions caused by Yersinia pseudotuberculosis, e.g. by immunisation.

### SUMMARY OF THE INVENTION

The present invention is based on the discovery of virulence genes in Yersinia species, in particular, Yersinia pestis and Yersinia pseudotuberculosis.

According to a first aspect of the invention, a peptide of the invention is encoded by a gene comprising any of the nucleotide sequences identified herein as SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, or a homologue thereof in a Gram-negative bacterium having at least 60% sequence similarity or identity at the peptide or nucleotide level, or a functional fragment thereof, for therapeutic or diagnostic use.

The peptide has many therapeutic uses for treating Yersinia infections, including use in vaccines for prophylactic application.

According to a second aspect of the invention, a polynucleotide encoding a peptide defined above, is also useful for therapy or diagnosis.

According to a third aspect of the invention, a gene that encodes the peptide is utilised to prepare an attenuated microorganism. The attenuated microorganism has a mutation that disrupts the expression of a gene identified herein, to provide a strain that lacks virulence. This microorganism will also have use in therapy and diagnosis.

According to a fourth aspect of the invention, a peptide, gene or attenuated microorganism of the invention is used in the preparation of a medicament for the treatment or prevention of a condition associated with infection by Yersinia or Gram-negative bacteria, e.g. gastroenteritis.

According to a fifth aspect of the invention, a vaccine comprises a peptide of the invention, in a suitable diluent, excipient or pharmacologically acceptable buffer. The vaccine is used in therapy to treat or prevent infection by Yersinia or Gram-negative bacteria.

According to a sixth aspect of the invention, an antibody is raised against a peptide of the invention. The antibody can be used in immunotherapy to treat infection.

According to a seventh aspect of the invention, a peptide, polynucleotide or microorganism of the invention is used in an assay to screen for potential antimicrobial drugs.

### DESCRIPTION OF THE INVENTION

The present invention is based on the discovery of genes encoding peptides which are implicated in virulence. A peptide and gene of the invention is therefore useful for the preparation of therapeutic agents to treat infection. It should be understood that references to therapy also include preventative treatments, e.g. vaccination. Furthermore, while the products of the invention are intended primarily for treatment of infections in human patients, veterinary applications are also considered to be within the scope of the invention.

The present invention is described with reference to Yersinia pseudotuberculosis. However, all the Yersinia strains, and many other Gram-negative bacterial strains, are likely to include related peptides or proteins having amino acid sequence identity or similarity to those identified herein. Organisms likely to contain the peptide include, but are not limited to the genera Salmonella, Enterobacter, Klebsiella, Shigella and Yersinia.

In a preferred embodiment, the peptides comprise the Yersinia pseudotuberculosis amino acid sequence that corresponds to that disclosed herein for Yersinia pestis.

Preferably, the peptides that may be useful in the various aspects of the invention have greater than a 60% similarity with the peptides identified herein. More preferably, the peptides have greater than 80% sequence similarity. Most preferably, the peptides have greater than 90% sequence similarity, e.g. 95% similarity. With regard to the polynucleotide sequences identified herein, related polynucleotides that may be useful in the various aspects of the invention have greater than 60% identity with the sequences identified herein. More preferably, the polynucleotide sequences have greater than 80% sequence identity. Most preferably, the polynucleotide sequences have greater than 90% sequence identity, e.g. 95% identity.

The terms “similarity” and “identity” are known in the art. The use of the term “identity” refers to a sequence comparison based on identical matches between correspondingly identical positions in the sequences being compared. The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

Levels of identity between gene sequences and levels of identity or similarity between amino acid sequences can be calculated using known methods. In relation to the present invention, publicly available computer based methods for determining identity and similarity include the BLASTP, BLASTN and FASTA (Atschul et al., J. Molec. Biol., 1990; 215:403-410), the BLASTX program available from NCBl, and the Gap program from Genetics Computer Group, Madison Wis. The levels of similarity and. identity provided herein, were obtained using the Gap program, with a Gap penalty of 12 and a Gap length penalty of 4 for determining the amino acid sequence comparisons, and a Gap penalty of 50 and a Gap length penalty of 3 for the polynucleotide sequence comparisons.

Having characterised a gene according to the invention, it is possible to use the gene sequence to search for related genes or peptides in other microorganisms. This may be carried out by searching in existing databases, e.g. EMBL or GenBank.

Peptides or proteins according to the invention may be purified and isolated by methods known in the art. In particular, having identified a gene sequence, it will be possible to use recombinant techniques to express the gene in a suitable host. Active fragments and related molecules can be identified and may be useful in therapy. For example, a peptide or its active fragment may be used as an antigenic determinant in a vaccine, to elicit an immune response. They may also be used in the preparation of antibodies, for passive immunisation, or diagnostic applications. Suitable antibodies include monoclonal antibodies, or fragments thereof, including single-chain Fv fragments. Methods for the preparation of antibodies will be apparent to those skilled in the art.

Active fragments are those that retain a biological function of the peptide or which generate antibodies that are specific for that peptide. For example, when used to elicit an immune response, the fragment will be of sufficient size, such that antibodies generated from the fragment will discriminate between that peptide and other peptides of the bacterial microorganism. Typically, the fragment will be at least 30 nucleotides (10 amino acids) in size, preferably 60 nucleotides (20 amino acids) and most preferably greater than 90 nucleotides (30 amino acids) in size.

It should also be understood, that in addition to related molecules from other microorganisms, the invention encompasses modifications made to the peptide and polynucleotide identified herein which do not significantly alter its biological role. It will be apparent to the skilled person that the degeneracy of the genetic code can result in polynucleotides with minor base changes from those specified herein, but which nevertheless encode the same peptide. Complementary polynucleotides are also within the invention. Conservative replacements at the amino acid level are also envisaged, i.e. different acidic or basic amino acids may be substituted without substantial loss of function.

Included within the scope of the claimed invention are molecules that comprise a polynucleotide which hybridizes under stringent hybridization conditions to a portion of a polynucleotide of the invention. By “stringent hybridization conditions” is intended overnight incubation at 42° C. in a solution comprising: 50% formamide, 5×SSC (150 nM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

By a “polynucleotide which hybridizes to a portion” of a polynucleotide is intended a polynucleotide (either DNA or RNA) hybridization to at least 15 nucleotide bases, and more preferably at least 20 nucleotide bases, still more preferably at least 30 nucleotide bases, and even more preferably 30-70 (e.g. 50) nucleotide bases of the reference polynucleotide.

The preparation of vaccines based on attenuated microorganisms is known to those skilled in the art. Vaccine compositions can be formulated with suitable carriers or adjuvants, e.g. alum, as necessary or desired, to provide effective immunisation against infection. The preparation of vaccine formulations will be apparent to the skilled person. The attenuated microorganisms may be prepared with a mutation that disrupts the expression of a gene identified herein. The skilled person will be aware of methods for disrupting expression of particular genes. Techniques that may be used include insertional inactivation or gene deletion techniques. Attenuated microorganisms according to the invention may also comprise additional mutations in other genes, for example in a gene required for growth of the microorganism, e.g. an aro mutation.

Attenuated microorganisms may also be used as carrier systems for the delivery of heterologous antigens, therapeutic proteins or nucleic acids (DNA or RNA). In this embodiment, the attenuated microorganisms are used to deliver a heterologous antigen, protein or nucleic acid to a particular site in vivo. Introduction of a heterologous antigen, peptide or nucleic acid into an attenuated microorganism can be carried out by conventional techniques, including the use of recombinant constructs, e.g. vectors, which comprise polynucleotides that express the heterologous antigen or therapeutic protein, and also include a suitable promoter sequence. Alternatively, the gene that encodes the heterologous antigen or protein may be incorporated into the genome of the organism and an endogenous promoter used to control expression.

More generally, and as is well known to those skilled in the art, a suitable amount of an active component of the invention can be selected, for therapeutic use, as can suitable carriers or excipients, and routes of administration. These factors will be chosen or determined according to known criteria such as the nature/severity of the condition to be treated, the type and/or health of the subject etc.

In a separate embodiment, the products of the invention may be used in screening assays for the identification of potential antimicrobial drugs or for the detection for virulence. Routine screening assays are known to those skilled in the art, and can be adapted using the products of the invention in the appropriate way. For example, the products of the invention may be used as the target for a potential drug, with the ability of the drug to inactivate or bind to the target indicating its potential antimicrobial activity.

The various products of the invention may also be used in veterinary applications.

The following is a summary of the experimental procedure used to identify the virulence genes. The full experimental procedure and results have now been published in Karlyshev et al., Infection and Immunity, 2001; 69(12): 7810-7819.

The virulence genes of the invention were identified using a modified version of the signature-tagged mutagenesis (STM) method (Hensel et al., Science, 1995; 269: 400-403), to screen a Yersinia pseudotuberculosis mutant bank for attenuated mutants, in a murine model of Yersiniosis infection. Bacteria containing a transposon insertion within a virulence gene failed to be recovered from mice inoculated with a mixed population of mutants.

The transposons used in the method contained DNA tages that were amplified using biotinylated primers and hybridised to high-density oligonucleotide arrays containing DNA complementary to the tags. Comparison of the hybridisation signals from input pools and output pools identified mutants whose relative abundance was significantly reduced in the output pool.

The sequence data from the transposon insertion regions was then compared to the complete Yersinia pestis C092 genome sequence.

Bacterial Strains and Growth Conditions:

Y. pseudotuberculosis YPIII plB1 strain (Rosquist et al., Nature, 1988; 334:522-525) was maintained in Luria Broth (LB) and LB agar containing nalidixic acid (40 μg ml⁻¹). E. coli XL2 Blue MRF′ (Stratagene), used in cloning experiments, were grown overnight at 37° C. on LB agar plates. For selection, agar plates were supplemented with the antibiotics kanamycin (50 μg ml⁻¹), ampicillin (100 μg ml⁻¹), tetracycline (10 μg ml⁻¹) or nalidixic acid. E. coliCC118(λpir) (Herrero et al., J. Bacteriol, 1990; 172:6557-6567) was used as a host strain for maintenance of the pir-dependent pUT mini-Tn5Km2 vector (de Lorenzo et al., J. Bacteriol., 1990; 172: 6568-6572) in cloning experiments. The helper strain E. coli S17/pNJ5000 was maintained as described in Grinter et al., Gene, 1983; 21: 133-143.

Construction of Double-Tagged Mini-Tn5 Transposon Mutants:

Tag-sequences were chosen from those that had been shown to work well in similar experiments with Saccharomyces cerevisiae (Winzeler et al., Science, 1999; 285: 901-906). The sequences of the 192 PCR primers (primer A and primer B) and the preparation of plasmids carrying tagged mini-Tn5 transposons are shown in Karlyshev et al., 2001, supra.

Conjugation:

Initial triple mating experiments of E. coli CC118 (λpir) donor strain, transformed with the plasmids carrying tagged mini-Tn5, and Y. pseudotuberculosis using a helper strain E. coli S17/pNJ5000 (Grinter et al., 1984, supra) were performed as described in Oyston et al., Microbiology, 1996; 142: 1847-1853. Direct mating experiments (without a helper strain) using E. coli 19851 pir⁺ as the donor strain were performed as described in Metcalf et al., Plasmid, 1996; 35: 1-13. Exconjugants were selected for kanamycin and nalidixic acid resistance. Both the recipient strairi, YPIII plB1, and the exconjugants were checked for the presence of the virulence plasmid using Congo red magnesium oxalate (CRMOX) plates (Riley et al., J. Clin. Microbiol., 1989; 27:213-214. All attenuated transposon derivatives grew as predominantly small red colonies, confirming that they retained the virulence plasmid.

Tag Sequence Detection:

Genomic DNA was extracted and the tags identified according to the protocol in Karlyshev et al., 2001, supra.

In Vivo Experiments:

Three input pools containing 60, 40 and 33 transposon mutants respectively, were constructed and stored at −70+ C. Aliquots (0.1 ml) containing approximately 10⁷ cfu were inoculated into 10 ml LB and incubated with shaking overnight at 30° C. The overnight culture (2 ml) was used to inoculate 20 ml of fresh pre-warmed LB and further incubated at 37° C. for 3 hours with shaking.

Genomic DNA was isolated from approximately 10⁸ cells and stored (input pool). Bacteria were pelleted at 3,000×g and diluted in Phosphate buffered saline (PBS) for infection and viable count determination. Pairs of eight-week-old female Balb/c mice were challenged intravenously (iv) via the tail vein with 10⁵ or 5×10⁵ cfu. After 3 days, the surviving mice were culled, spleens were removed and homogenized in 3 ml of LB using a stomacher (Seward Medical Ltd) on maximum setting for 5 minutes. Dilutions of the extracts were plated on LB agar containing kanamycin and nalidixic acid. Plates containing approximately 10⁴ colonies were washed with saline, mixed and aliquots were taken for making lysates (for PCR) or for total DNA preparation. Genomic DNA recovered from the spleens were the output pools.

Mutants with reduced survival in vivo were visualised by comparing the scanned images from arrays that had been hybridized with tags amplified from the Input pools with images obtained from two independent output pools.

The input and output pools of the mutants were compared by hybridizing the labeled amplified tags to high-density oligonucleotide arrays (Affymetrix) containing complementary DNA sequences. The hybridization patterns were found to be reproducible. Mutants that showed reduced signals in the output pool for both tags in duplicate mice were selected for further analysis.

Characterization of Attenuated Mutants and Identification of the Transposon Insertion Sequences:

Approximately 5% out of 603 exconjugants exhibited a statistically valid reduction of signal intensity. Transposon insertion sites in the selected mutants were sequenced using a single primer PCR sequencing procedure (Karlyshev et al., BioTechniques, 2000; 28: 1078-1082). The results are summarized in Tables 1 and 2. The Y. pestis genome sequence database (http://www.sanger.ac.uk/Projects/Y_{—}pestisl) was used for identification of the corresponding genes in that pathogen; almost 100% identity of the Y. pseudotuberculosis sequences to the Y. pestis DNA sequences implies similarity in their function. The YPO number is the accession number that is used to identify the gene in the Y. pestis genome sequence database. The SEQ ID NOS. 1-58 and 64 are the Yersinia pestis sequences. SEQ ID NOS. 35, 56, 57 and 58 are genes in Y. pestis that appear to be non-functional in that they appear to contain many mutations in the gene sequence that disrupt the expression of an amino acid product. However, the orthologue (homologue) in Y. pseudotuberculosis is expected to be functional. The reference to “nrdb” refers the non-redundant amino acid database (www.blast.genome.ad.jp). Any orthologue found in this database is indicated in the columns to the right of the nrdb value.

Certain gene sequences had no orthologue in Y. pestis. These genes are identified herein by the mutant number 5D12, SH10, 5B12, lAg-1 and 1C9. The sequence provided herein for these mutants is not the complete gene sequence but is the flanking sequence of the transposon insertion site in Yersinia pseudotuberculosis. This sequence may not be part of the virulence gene but may be an upstream regulatory site. The flanking sequence is used to identify a suitable site for mutation that will result in a loss of virulence in the microorganism. Accordingly, mutant microorganisms can be prepared which have an attenuating mutation within the sequence identified herein.

In addition to the preparation of attenuated microorganisms, the encoded products of the genes identified herein are suitable as targets for immunotherapy oras immunogenic components of vaccines. In this context, the products identified by the references 5E4, 2G8, 5G6, 1D12, 5G7, 1A9, 4H2, 3G1, 5A5, 3F10, 2B3, 1H6, 2G5, 3G6, 2G10, 1 H9, 4F4 and 4G11, are all preferred as they are located on the outer or inner membrane, or are extracellular proteins, shown in Table 3. In Table 3, IM, PP, OM and EC denote inner membrane, periplasmic, outer membrane and extracellular, respectively.

TABLE 1

| | SEQ | In | In | Similarity, | | Similarity, | | |
|---|---|---|---|---|---|---|---|---|
| | ID | vivo | vitro | Y. pestis | Y. pestis | nrdb | | |
| Mutant | NO. | Cl | Cl | (%) | ORF | (%) | Product | Microorganism |
| 4H9 | 1 | 0.03 | 0.06 | 97 | YPO0054 | 85 | glycosyltransferase | S. marcescens |
| 2B3 | 3 | 1.02 | 85 | 94 | YPO0337 | NS | — | — |
| 5E4 | 5 | NT | NT | 99 | YPO0702 | 46 | putative lipoprotein | Mycobacterium |
| | | | | | | | | tuberculosis |
| 30 | 7 | 0.48 | 0.31 | 98 | YPO1108 | 91 | citrate synthase | S. typhimurium |
| 2G8 | 9 | 0.53 | 1.34 | 96 | YPO1174 | 39 | Adhesin | E. coli |
| 3C10 | 11 | 0.055 | 1.02 | 98 | YPO1186 | 66 | hypothetical | E. coli |
| 3H10 | 13 | 0.08 | 0.07 | 97 | YPO1382 | 43 | LpsA, | Pasteurella |
| | | | | | (promoter | | glycosyltransferase | haemolytica |
| | | | | | region) | | | |
| 5G6 | 15 | 0.021 | 0.88 | 95 | YPO1987 | NS | — | — |
| 1D12 | 17 | 0.084 | 1.34 | 97 | YPO1994 | NS | — | — |
| 1H10 | 19 | NT | NT | 98 | YPO2174 | 62 | UDP-glucose 6- | Rhizobium meliloti |
| | | | | | | | dehydrogenase | |
| 4H10 | 21 | 0.0036 | 1.95 | 96 | YPO2287a | 65 | ABC-transporter | Methanococcus |
| | | | | | | | | jannaschii |
| 5G7 | 23 | 0.25 | 1.09 | 98 | YPO2440 | 82 | iron(III) dicitrate | Haemophilus |
| | | | | | | | ATP-binding protein | influenzae |
| 1A9 | 25 | NT | NT | 100 | YPO2532 | NS | — | — |
| 4H2 | 27 | NT | NT | 98 | YPO2712 | 77 | RseA, negative | E. coli |
| | | | | | | | regulator of RpoE | |
| | | | | | | | (sigma 24) | |
| 5E6 | 56 | 0.27 | 1.24 | 97 | YPO3004 | 47 | Pro-dipeptidase | B. subtilis |
| 1D2 | 29 | 0.43 | 0.08 | 96 | YPO3099 | 100 | ManC, mannose-1-P | Y. pseudotuberculosis |
| | | | | | | | guanylyltransferase | |
| 3G2 | 57 | 0.13 | NT | 98 | YPO3100 | 100 | Fcl, fucose | Y. pseudotuberculosis |
| | | | | | | | synthetase | |
| 1B3 | 31 | 0.29 | 0.98 | 90 | YPO3104 | 100 | O-antigen | Y. pseudotuberculosis |
| | | | | | | | polymerase | |
| 1D9 | 58 | NT | 0.07 | 98 | YPO3114 | 100 | DdhB, CDP-D- | Y. pseudotuberculosis |
| | | | | | | | glucose-dehydratase | |
| 3F3 | 33 | 0.04 | 0.44 | 95 | YPO3116 | 100 | AscD, ascarilose | Y. pseudotuberculosis |
| | | | | | | | biosynthesis | |
| 3G1 | 35 | 0.21 | 1.25 | 97 | YPO3144 | 89 | MdlB, Multi-drug | E. coli |
| | | | | | | | resistance protein | |
| 5A5 | 36 | 0.44 | 1.16 | 98 | YPO3572 | 88 | hypothetical | E. coli |
| | | | | | | | transcription factor | |
| 1G6 | 38 | NT | NT | 97 | YPO3657 | NS | Unknown | — |
| | | | | | (intergenic) | | | |
| 3F10 | 40 | 0.017 | 0.46 | 99 | YPO3834 | 87 | PldA, phospholipase A | E. coli |
| 1G6-1 | 42 | NT | 0.41 | 96 | YPO3965 | 47 | VirA, His kinase | Agrobacterium |
| | | | | | | | | tumefaciens |
| 5D12 | 59 | 0.003 | 0.98 | NS | — | 80 | Wzx | S. typhimurium |
| 5H10 | 60 | NT | NT | NS | — | 70 | phage-related | Xylella fastidiosa |
| | | | | | | | transcription | |
| | | | | | | | activator | |
| 5B12 | 61 | 0.89 | 1.32 | NS | — | 59 | hypothetical | phage HP1 |
| 1A9-1 | 62 | NT | NT | NS | — | NS | Unknown | — |
| 1C9 | 63 | NT | 0.36 | NS | — | NS | Unknown | — |
| 1G6a | 64 | NT | NT | 100 | YPO03657a | — | YhdT | E. coli |

TABLE 2

| Mutant | SEQ ID NO. | Orthologue in Y. Pestis |
|---|---|---|
| 2G5 | 44 | YPO1485 |
| 3G6 | 46 | YPO2503 |
| 2G10 | 48 | YPO1483 |
| 1H9 | 50 | YPO1696 |
| 4F4 | 54 | YPO0036 |
| 4G11 | 52 | YPO3010 |

TABLE 3

| | Orthologue in | | Gene | YP product | subcellular |
|---|---|---|---|---|---|
| Mutant | Y. pestis | Orthologue in nrdb | name | size, aa | location¹ |
| 1H6 | YPO3965 | VirA, Agrobacterium | virA | 851 | IM |
| | | tumefaciens, 9e−30 | | | |
| 3F10 | YPO3834 | PldA, S. typhimurium, | pldA | 292 | OM |
| | | e−127 | | | |
| 2G8 | YPO1174 | low score similarities to C- | — | 309 | OM |
| | | term domains of various | | | |
| | | OM proteins | | | |
| 5G7 | YPO2440 | YfeB, H. influenzae, | yfeB | 296 | IM |
| | | e−111 | | | |
| 3G1 | YPO3144 | MdlB, E. coli, | MdlB | 607 | IM |
| | | e0.0 | | | |
| 2B3 | YPO0337 | no simil. | — | 141 | OM |
| 5G6 | YPO1987 | putative PP of S. typhimurium, | — | 552 | ? |
| | | e−146 | | | |
| 1D12 | YPO1994 | no simil. | — | 316 | ? |
| 5A5 | YPO3572 | Putative EC protein of S. enterica, | — | 207 | PP or OM |
| | | 2e−91 | | | |
| 4H2 | YPO2712 | RseA | rseA | 218 | |
| 1A9 | YPO2532 | no simil. | — | — | ? |
| 5E4 | YPO0702 | low similarity to various | | 331 | OM or PP |
| | | putative secreted proteins of | | | |
| | | the same size | | | |
| 2G5 | YPO1485 | hyp. protein VCA0110 | — | 587 | ? |
| | | V. cholerae, | | | |
| | | 3e−66 | | | |
| 3G6 | YPO2503 | no simil. | — | 222 | ? |
| 2G10 | YPO1483 | no simil. | — | 533 | ? |
| 1H9 | YPO1696 | various usher proteins | — | 815 | OM |
| 4F4 | YopH | YopH | yopH | 468 | EC |
| 4G11 | YPO3010 | YopM Y. ent. | yopM-2 | 410 | EC |

Genes Involved in Polysaccharide Biosynthesis:

One third of the sequenced mutants had transposon insertions in genes related to polysaccharide biosynthesis (mainly LPS core or O-antigen biosynthesis). In five cases (1B3, 1D2, 1D9, 3G2 and 3F3; Table 1) the genes disrupted belong to a single characterized O-antigen biosynthesis locus of Y. pseudotuberculosis. The disrupted genes encode mannose-phosphate-guanylyl transferase YPO3099 (1D2), fucose synthetase YPO3100 (3G2), LPS core biosynthesis protein YPO3104 (1B3), sugar dehydratase YPO3114 (1D9) and ascarylose biosynthesis protein YPO3116 (3F3). These genes are also present in Y. pestis. Other genes related to polysaccharide biosynthesis, such as those encoding UDP-glucose 6-dehydrogenase (1H10) and glycosyltransferase (4H9) were also identified. One mutant,3H10, contained an insert in a putative promoter region of a single-gene operon encoding glycosyltransferase. The only mutation in an LPS-related gene which was absent in Y. pestis encoded an O-antigen transporter (5D12).

Putative Virulence-Related Genes with Orthologues in Other Bacteria:

Putative interrupted virulence genes include those encoding phospholipase A (pldA) (3F10), sensory transducer His kinase VirA (1G6-1), a putative adhesin (2G8), a Pro-dipeptidase (5E6); RseA, a negative regulator of sigma 24 transcription factor (4H2) a transcription activator (5H10) and a transcription regulator (5A5) flanked by a vspC gene essential for secretion of virulence factors. The genes found in 4H10 and 3G1 are related to ABC transporters, a large class of proteins involved in export-import of a range of molecules. A clue to their possible function can be found from the analysis of corresponding regions of Y. pestis. In the case of 3G1, other genes of the operon are involved in ammonia assimilation. These genes are assumed to be essential in vivo, when bacteria have a depleted source of nitrogen. Similarly, the Y. pestis orthologue of the gene disrupted in the ABC transporter (4H10) is also located in the region involved in nitrate, as well as amino acid, transport. An unusual feature of this region in Y. pestis is that this gene overlaps by 270 nucleotides with another gene (AMP nucleosidase) transcribed in the opposite direction. Disrupted genes in the iron-IIII dicitrate ATP-binding protein 5G7 is likely to be involved in iron transport.

Unknown and Hypothetical Genes:

Six mutants (1G6, 5D12, 5H10, 5B12, 1A9-1 and 1C9) contained inserts into genes with unknown function. Two of them (1C9 and 1A9-1) did not have counterparts in Y. pestis. Both these mutants contained inserts in the region identical to an E. coli integrase-recombinase pseudogene, but actually had inserts into an adjacent region similar to an E. coli gene with unknown function. Transposon integration sites in these mutants were separated by approximately 200 nucleotides. A region flanking the insertion site in the 1G6 appeared to be unique for Yersinia as it had 97% identity in the Y. pestis database, whereas no similarity to other bacteria was found. Genes in mutants 3C10 and 5B12 have similarities in a non-redundant database (nrdb) and also have counterparts in Y. pestis. The genes inactivated in mutants 2B3, 5G6, 1D12 and ,1A9 appear to be unique for Yersinia, as no homoidgues could be identified in a nrdb. Genes inactivated in certain mutants do not have Y. pestis orthologues. One of these mutants (5D12) contains insert in the gene encoding a putative O-antigen transferase (see above).

In Vivo and in Vitro Competition Studies:

For in vivo competition studies, mutant and wild-type strains were grown separately to exponential phase in LB media with appropriate antibiotics. Bacteria were. washed with LB media and the concentration adjusted to 5×10⁶ cfu/ml. Equal volumes of each bacterial suspension were mixed together and 0.1 ml volumes were injected iv into 4 mice as above. Viable counts on LB, LB-Nal and LB-Nal-Kan allowed the exact input ratio to be calculated. After 3 days, spleens were recovered and passed through sieves (70 μm, Becton Dickinson) to produce a cell suspension in 3 ml LB. Homogenates were plated on selective media to determine the output ratio. The competitive index is defined as the output ratio (mutant/wt) divided by the input ratio (mutant/wt).

In-vitro Cl was determined as described in Chiang et al., Mol. Microbiol., 1998; 27: 797-805. Briefly, mixtures containing a mutant and the wild-type strains were inoculated into LB media supplemented with nalidixic acid at approximately 1×10⁴ cfu/ml. The cultures were grown overnight at 28° C. and the mutant to wild-type ratios were determined by plating on media with and without selective marker (kanamycin).

The majority of the selected mutants with reduced output signals did not reveal significant reduction in in vitro growth properties and were confirmed to be attenuated (Table 1). Four mutants (1D9, 1D2, 4H9 and 3H10) revealed substantial (more than six times) reduction in Cl. In all cases, the genes affected are related to LPS or LPS core biosynthesis. For example, an orthologue to a gene inactivated in 4H10 is located in an operon containing a number of other genes, such as kdtB, waaA, rfaC, rfaD and rfaF, all related to core biosyntheses. The insert in mutant3H10 would also inactivate a gene encoding an LPS core-related glycosyltransferase. Inactivation of genes in other mutants of this class may have a dramatic effect on outer membrane stability due to an affect on LPS biosynthesis.

In vivo Cl figures of less than 0.3 were demonstrated in 14 out of 20 cases, confirming attenuated properties of these mutants.

In vivo competition studies revealed significant attenuation of the 3F10 (PldA) derivative. The competitive index (Cl=0.01655) obtained in the mixed infection experiment confirmed that the strain was severely attenuated. These data suggest that PldA is an essential virulence factor in the murine yersiniosis model of infection.

1-19. (canceled)

20. A peptide encoded by a gene wherein said gene comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof.

21. The peptide according to claim 20, wherein the sequence similarity or identity is at least 80%.

22. The peptide according to claim 20, wherein the sequence similarity or identity is at least 90%.

23. The peptide according to claim 20, comprising an amino acid sequence selected from the group consisting of SEQ ID NOS. 2, 4, 6, 8, 10, 12, 14, 16, 18, 20, 22, 24, 26, 28, 30, 32, 34, 37, 39, 41, 43, 45, 47, 49, 51, 53, 55 and 65.

24. A polynucleotide encoding a peptide wherein said polynucleotide comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof.

25. A polynucleotide encoding a peptide, wherein said peptide comprises an amino acid sequence selected from the group consisting of SEQ ID NOS. 2, 4, 6, 8, 10, 12, 14, 16, 18, 20, 22, 24, 26, 28, 30, 32, 34, 37, 39, 41, 43, 45, 47, 49, 51, 53, 55 and 65.

26. A recombinant host genetically modified to express a peptide encoded by a gene comprising a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof.

27. An attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58, 59, 60, 61, 62, 63, and 64, of Yersinia pseudotuberculosis, related sequences obtainable from Gram-negative bacteria that have at least 60% sequence similarity or identity, and functional fragments thereof.

28. The microorganism according to claim 27, wherein the mutation is insertional inactivation or a gene deletion.

29. The microorganism according to claim 27, wherein the microorganism is Yersinia pseudotuberculosis or Yersinia pestis.

30. The microorganism according to claim 27, comprising a second mutation in a second nucleotide sequence.

31. The microorganism according to claim 27, comprising a heterologous antigen, therapeutic peptide or nucleic acid.

32. A vaccine comprising a peptide encoded by a gene, wherein said gene comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof.

33. The vaccine according to claim 32, wherein said peptide comprises an amino acid sequence selected from the group consisting of SEQ ID NOS. 2, 4, 6, 8, 10, 12, 14, 16, 18, 20, 22, 24, 26, 28, 30, 32, 34, 37, 39, 41, 43, 45, 47, 49, 51, 53, 55, and 65.

34. A vaccine comprising an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58, 59, 60, 61, 62, 63, and 64, of Yersinia pseudotuberculosis, related sequences obtainable from Gram-negative bacteria that have at least 60% sequence similarity or identity, and functional fragments thereof.

35. An antibody raised against a peptide encoded by a gene, wherein said gene comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof.

36. The antibody according to claim 35, wherein said peptide comprises an amino acid sequence selected from the group consisting of SEQ ID NOS. 2, 4, 6, 8, 10, 12, 14, 16, 18, 20, 22, 24, 26, 28, 30, 32, 34, 37, 39, 41, 43, 45, 47, 49, 51, 53, 55, and 65.

37. A method for the treatment or prevention of a condition associated with infection by Yersinia, wherein said method comprises administering to a patient in need of such treatment or prevention a compound selected from one or more of the following:
(i) a peptide encoded by a gene wherein said gene comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof;
(ii) a peptide comprising an amino acid sequence selected from the group consisting of SEQ ID NOS. 2, 4, 6, 8, 10, 12, 14, 16, 18, 20, 22, 24, 26, 28, 30, 32, 34, 37, 39, 41, 43, 45, 47, 49, 51, 53, 55 and 65;
(iii) a polynucleotide encoding a peptide wherein said polynucleotide comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof;
(iv) a recombinant host genetically modified to express a peptide encoded by a gene comprising a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof;
(v) an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58, 59, 60, 61, 62, 63, and 64, of Yersinia pseudotuberculosis, related sequences obtainable from Gram-negative bacteria that have at least 60% sequence similarity or identity, and functional fragments thereof;
(vi) a vaccine comprising a peptide encoded by a gene, wherein said gene comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof; and
(vii) a vaccine comprising an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58, 59, 60, 61, 62, 63, and 64, of Yersinia pseudotuberculosis, related sequences obtainable from Gram-negative bacteria that have at least 60% sequence similarity or identity, and functional fragments thereof.

38. The method according to claim 37, wherein the condition is tuberculosis.

39. The method according to claim 37, for treating an animal.

40. A screening assay for the identification of an antimicrobial drug wherein said assay uses one or more of the following:
(i) a peptide encoded by a gene wherein said gene comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof;
(ii) a peptide comprising an amino acid sequence selected from the group consisting of SEQ ID NOS. 2, 4, 6, 8, 10, 12, 14, 16, 18, 20, 22, 24, 26, 28, 30, 32, 34, 37, 39, 41, 43, 45, 47, 49, 51, 53, 55 and 65;
(iii) a polynucleotide encoding a peptide wherein said polynucleotide comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof, and
(iv) an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58, 59, 60, 61, 62, 63, and 64, of Yersinia pseudotuberculosis, related sequences obtainable from Gram-negative bacteria that have at least 60% sequence similarity or identity, and functional fragments thereof.

41. A method for the manufacture of a medicament for use in the treatment or prevention of a condition associated with infection by a Yersinia species, wherein said method utilizes at least one or more of the following:
(i) a peptide encoded by a gene wherein said gene comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof;
(ii) a peptide comprising an amino acid sequence selected from the group consisting of SEQ ID NOS. 2, 4, 6, 8, 10, 12, 14, 16, 18, 20, 22, 24, 26, 28, 30, 32, 34, 37, 39, 41, 43, 45, 47, 49, 51, 53, 55 and 65;
(iii) a polynucleotide encoding a peptide wherein said polynucleotide comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof;
(iv) a recombinant host genetically modified to express a peptide encoded by a gene comprising a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof;
(v) an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58, 59, 60, 61, 62, 63, and 64, of Yersinia pseudotuberculosis, related sequences obtainable from Gram-negative bacteria that have at least 60% sequence similarity or identity, and functional fragments thereof;
(vi) a vaccine comprising a peptide encoded by a gene, wherein said gene comprises a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58 and 64, of Yersinia pseudotuberculosis, related sequences obtainable from Gram-negative bacteria that have at least 60% sequence similarity or identity, and functional fragments thereof; and
(vii) a vaccine comprising an attenuated microorganism comprising a mutation that disrupts the expression of a nucleotide sequence selected from the group consisting of SEQ ID NOS. 1, 3, 5, 7, 9, 11, 13, 15, 17, 19, 21, 23, 25, 27, 29, 31, 33, 35, 36, 38, 40, 42, 44, 46, 48, 50, 52, 54, 56, 57, 58, 59, 60, 61, 62, 63, and 64, of Yersinia pseudotuberculosis, related sequences obtainable from Gram-negative bacteria that have at least 60% sequence similarity or identity, and functional fragments thereof.
